(19) United States
(12) Patent Application Publication
(10) Pub. No.: US 2014/0134745 A1
(43) Pub. Date: May 15, 2014

(54) METHOD FOR EVALUATION OF LIGNIN

(71) Applicants: John R. Peterson, Chardon, OH (US); Vladimir Romakh, Mississauga (CA); Jian Wu, Guelph (CA)

(72) Inventors: John R. Peterson, Chardon, OH (US); Vladimir Romakh, Mississauga (CA); Jian Wu, Guelph (CA)

(73) Assignee: VERTICHEM CORPORATION, Cambridge (CA)

(21) Appl. No.: 13/677,702

(22) Filed: Nov. 15, 2012

(51) Int. Cl. G01N 33/00; G06F 17/18

(52) U.S. Cl. CPC G01N 33/0098; G06F 17/18
USPC 436/93; 73/866; 702/27

(57) ABSTRACT

According to one aspect of the present method, a method for evaluation of lignin may comprise the steps of: providing lignin from a source, wherein the composition of lignin is unknown; analyzing the lignin by molecular spectroscopy methods and/or physical-chemical analysis; determining the composition of the lignin; and using the determination of the composition to predict at least one biobased product produced from the lignin. Within the method, a statistical analysis of the lignin may provide a prediction for one or more products produced and/or the yield of one or more products.

### I. BACKGROUND

A. Field

Described herein is a method for the evaluation of lignin to determine its composition. From this analysis, at least one biobased product produced from lignin may be predicted.

B. Description of the Related Art

Biomass is made up primarily of cellulose, hemicellulose, and lignin. These components, if economically separated from one another, can provide sources of chemicals normally derived from petrochemicals. The use of biomass can also be beneficial with agricultural and/or woody plants that are sparsely used and plant wastes that currently have little or no use. Biomass can provide valuable chemicals and reduce dependence on coal, gas, and fossil fuels, in addition to boosting local and worldwide economies. In processes separating biomass, several options are available: the OrganoSolv™ and Alcell® processes which are used to efficiently remove the lignin from the other components under mild conditions, kraft pulping, sulfite pulping, pyrolysis, steam explosion, ammonia fiber explosion, dilute acid hydrolysis, alkaline hydrolysis, alkaline oxidative treatment, and enzymatic treatment.

Although the cellulosic fraction of biomass has garnered substantial attention recently as a feedstock for ethanol biofuel and other basic chemicals, the intrinsic value of the lignin continues to be largely overlooked. Lignin, which can comprise about 15% to about 30% of the organic matrix of woody and agricultural biomass, is an abundant source of aromatic chemicals outside of crude oil. Lignin can be used in developing technologies that transform plant biomass into value-added aromatic chemicals and products.

### II. SUMMARY

According to one aspect of the present method, a method for evaluation of lignin may comprise the steps of: providing lignin from a source, wherein the composition of lignin is unknown; analyzing the lignin; determining the composition of the lignin; and using the determination of the composition to predict at least one biobased product produced from the lignin.

One object of the present method is to provide lignin from at least one biomass of plant biomass, woody plant biomass, agricultural plant biomass, and/or cultivated plant biomass.

Another object of the present method is to provide lignin from at least one biomass of fresh plant biomass, recovered plant biomass, pulp and paper mill biomass, cellulosic ethanol refinery biomass, sugar cane mill biomass, kraft pulp mill, sulfite pulp mill, soda pulp mill, cellulosic ethanol refinery, commercial plant biomass fractionator biomass, and/or lignin residues biomass.

Still another object of the present method is to provide lignin from waste lignin, wherein the waste lignin is provided from at least one waste lignin of recovered biomass waste lignin, kraft pulp mill waste lignin, sulfite pulp mill waste lignin, soda pulp mill waste lignin, cellulosic ethanol refinery waste lignin, commercial plant biomass fractionator waste lignin, and/or sugar cane mill waste lignin.

Yet another object of the present method is to provide the lignin comprising of at least one lignin building block of p-coumaryl alcohol, coniferyl alcohol, and sinapyl alcohol.

Another object of the present method is to provide lignin analysis by molecular spectroscopy.

Still another object of the present method is to provide molecular spectroscopy by at least one molecular spectroscopy of infrared spectroscopy, total reflectance infrared spectroscopy, Raman spectroscopy, ultraviolet-visible spectroscopy, luminescence spectroscopy, fluorescence spectroscopy, mass spectroscopy, diffuse reflectance spectroscopy, transient spectroscopy, homonuclear magnetic resonance spectroscopy, heteronuclear magnetic resonance spectroscopy, and/or two-dimensional nuclear magnetic resonance spectroscopy.

Yet another object of the present method is to provide molecular spectroscopy by at least two molecular spectroscopies of infrared spectroscopy, total reflectance infrared spectroscopy, Raman spectroscopy, ultraviolet-visible spectroscopy, luminescence spectroscopy, fluorescence spectroscopy, mass spectroscopy, diffuse reflectance spectroscopy, transient spectroscopy, homonuclear magnetic resonance spectroscopy, heteronuclear magnetic resonance spectroscopy, and/or two-dimensional nuclear magnetic resonance spectroscopy.

Still yet another object of the present method is to provide lignin analysis by a physical-chemical analysis.

One object of the present method is to provide a physical-chemical analysis by at least one physical-chemical analysis of appearance analysis, moisture content analysis, melting point analysis, melting point range analysis, molecular weight analysis, molecular weight distribution analysis, size exclusion chromatographic analysis, thin layer liquid chromatographic analysis, high performance liquid chromatographic analysis, gas chromatographic analysis, particle size analysis, chemical oxidation analysis, chemical derivative analysis, chemical methoxy content analysis, chemical degradation analysis, biodegradation analysis, photochemical degradation analysis, pH titration analysis, thermogravimetric analysis, differential scanning calorimetric analysis, dynamic mechanical analysis, rheological analysis, pyrolysis degradation analysis, residual carbohydrate analysis, residue on ignition analysis, heavy metals analysis, density analysis, x-ray diffraction analysis, x-ray powder analysis, and elemental analysis.

Another object of the present method is to provide a physical-chemical analysis by at least two physical-chemical analyses of appearance analysis, moisture content analysis, melting point analysis, melting point range analysis, molecular weight analysis, molecular weight distribution analysis, size exclusion chromatographic analysis, thin layer liquid chromatographic analysis, high performance liquid chromatographic analysis, gas chromatographic analysis, particle size analysis, chemical oxidation analysis, chemical derivative analysis, chemical methoxy content analysis, chemical degradation analysis, biodegradation analysis, photochemical degradation analysis, pH titration analysis, thermogravimetric analysis, differential scanning calorimetric analysis, dynamic mechanical analysis, rheological analysis, pyrolysis degradation analysis, residual carbohydrate analysis, residue on ignition analysis, heavy metals analysis, density analysis, x-ray diffraction analysis, x-ray powder analysis, and elemental analysis.

One object of the present method is to provide a lignin analysis with at least one experimental datum related to the composition of the lignin.

Still another object of the present method is to provide a lignin analysis with at least one experimental datum related to a composition of the lignin comprising p-coumaryl alcohol, coniferyl alcohol, and sinapyl alcohol.

Yet another object of the present method is to provide a lignin analysis with at least one experimental datum processed manually and/or electronically.

Another object of the present method is to provide a lignin analysis with at least two experimental data related to the composition of the lignin.

Yet another object of the present method is to provide a lignin analysis with at least two experimental data related to a composition of the lignin comprising p-coumaryl alcohol, coniferyl alcohol, and sinapyl alcohol.

Still yet another object of the present method is to provide a lignin analysis comprising the additional step of performing a statistical analysis of experimental data.

One object of the present method is to provide a statistical analysis of experimental data by at least one computational method of manual computation, spreadsheet computation, website computation, and/or statistical software computation.

Another object of the present method is to provide a statistical analysis of experimental data on a calculator or a computer.

Still another object of the present method is to provide the statistical analysis of experimental data comprising of a regression analysis of experimental data.

Yet another object of the present method is to provide the regression analysis of the experimental data by at least one regression analysis method of linear regression analysis and/or multivariant regression analysis.

Another object of the present method is to provide the regression analysis of the experimental data by linear least squares regression analysis.

Still yet another object of the present method is to provide the regression analysis of experimental data by at least one computational method of manual computation, spreadsheet computation, website computation, and/or statistical software computation.

One object of the present method is to provide the regression analysis of experimental data on a calculator or a computer.

Yet another object of the present method is to provide the step of using the determination of the composition to predict at least one biobased product produced from the lignin with an accuracy of about 70% to about 100%.

Still yet another object of the present method is to provide the step of using the determination of the composition to predict at least one biobased product produced from the lignin with an accuracy of about 85% to about 100%.

One object of the present method is to provide the lignin analysis with a prediction of a product yield of at least one biobased product produced from the lignin.

Another object of the present method is to provide the lignin analysis with a prediction of a product yield of at least two biobased products produced from the lignin.

Still another object of the present method is to provide the step of using the determination of the composition to predict a product yield of at least one biobased product produced from the lignin with an accuracy of about 70% to about 100%.

Yet another object of the present method is to provide the step of using the determination of the composition to predict a product yield of at least one biobased product produced from the lignin with an accuracy of about 85% to about 100%.

Still another object of the present method is to provide at least one biobased product produced from the lignin biomass is at least one product of biobased chemicals, biofuels, biobased materials, and/or lignin residues.

One object of the present method is to provide a method of lignin quality control in at least one operation of a biobased product refinery operation, commercial biomass fractionators operation, pulp/paper mills operation, cellulosic ethanol refineries operation, and/or sugar cane mill operations.

Another object of the present method is to provide a method for evaluation and processing of lignin which may comprise the steps of: providing lignin from at least two sources, wherein the composition of lignin is unknown; analyzing each lignin from at least two sources; determining the composition of each lignin from at least two sources; using the determination of the composition of each lignin from at least two sources to predict at least one biobased product; determining at least one of biobased product to produce; and blending lignin in defined proportions and amounts from at least two sources to produce at least one biobased product.

Yet another object of the present method is to provide a method for evaluation of biomass feedstock, which may comprise the steps of: providing lignin from a source comprising at least one biomass of woody plant biomass, agricultural plant biomass, cultivated plant biomass, kraft pulping biomass, sulfite pulping biomass, soda pulping biomass, cellulosic ethanol refinery biomass, sugarcane mill biomass, lignin residue biomass, and/or waste biomass, wherein the composition of the lignin is unknown; analyzing the lignin by molecular spectroscopy; analyzing the lignin biomass by physical-chemical analysis; providing experimental data related to the molecular structure of lignin; performing regression analysis of the experimental data; and determining the composition of lignin; wherein the step of determining the composition of the lignin provides a prediction of at least one biobased product produced from the lignin and the prediction has an accuracy of about 85% to about 100%.

Still other benefits and advantages of the method will become apparent to those skilled in the art to which it pertains upon a reading and understanding of the following detailed specification.

### III. BRIEF DESCRIPTION OF THE DRAWINGS

The method may take physical form in certain parts and arrangement of parts, and will be described in detail in this specification and illustrated in the accompanying drawings which form a part hereof and wherein:

FIG. 1 is a diagram schematically illustrating the present method.

FIG. 2 schematically illustrates one aspect of the present method.

FIG. 3 schematically illustrates one aspect of the present method.

FIG. 4 schematically illustrates one aspect of the present method.

FIG. 5 schematically illustrates another aspect of the present method.

FIG. 6 schematically illustrates one aspect of the present method.

FIG. 7 schematically illustrates one aspect of the present method.

FIG. 8 schematically illustrates one aspect of the present method.

FIG. 9 schematically illustrates one aspect of the present method.

FIG. 10 schematically illustrates one aspect of the present method.

FIG. 11 schematically illustrates one aspect of the present method.

FIG. 12 schematically illustrates one aspect of the present method.

FIG. 13 schematically illustrates one aspect of the present method.

### IV. DETAILED DESCRIPTION OF THE METHOD

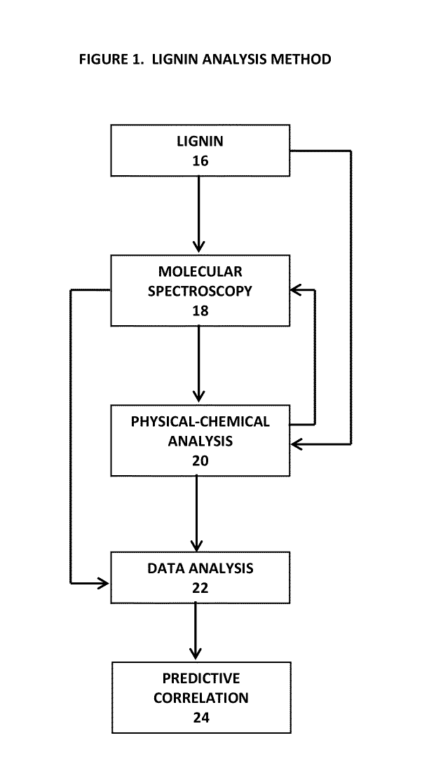

Referring now to the drawings, wherein the showings are for purposes of illustrating embodiments of the method only and not for purposes of limiting the same, FIGS. 1 through 13 aid in illustrating the method described herein.

The world currently faces depletion of fossil fuels while demands for these fuels are ever increasing. Petrochemicals provide an energy source and a component of the majority of raw materials used in many industries. In fact, approximately 95% of all chemicals manufactured today are derived from petroleum. However, this heavy reliance upon fossil fuels is creating harm to the environment. The burning of these fossil fuels has led to the pollution of air, water and land, as well as global warming and climate changes. Through the use of fossil fuels, the environment has been harmed, perhaps irreparably, in an effort to meet the nearly insatiable demand for energy and manufactured products. Fossil fuels are a finite natural resource. With the depletion of readily available oil reserves across the globe, the supply chain has shifted to more complex and environmentally risky production technologies. A reduction and conservation of fossil fuels is clearly needed. Some alternatives to fossil fuels, like solar power, wind power, geothermal power, hydropower, and nuclear power, are used to a degree. However, a more efficient use of renewable resources is always being sought.

As a stable and independent alternative to fossil fuels, biomass can be a potentially inexhaustible, domestic, natural resource for the production of energy, transportation fuels, and chemicals. The advantage in use of biomass for such purposes is magnified during an oil crisis, a surge in oil prices, or political unrest within oil producing regions of the world. Biomass includes plant and wood biomass, including agricultural biomass. Biomass can be employed as a sustainable source of energy and is a valuable alternative to fossil fuels. More specifically, the biorefining of biomass into derivative products typically produced from petroleum can help to stop the depletion of petroleum, or at least reduce the current demand and dependence. Biomass can become a key resource for chemical production in much of the world. Biomass, unlike petroleum, is renewable. Biomass can provide sustainable substitutes for petrochemically derived feedstocks used in existing markets.

Lignin has a complex, polymeric structure whose exact structure is unknown. This large group of aromatic polymers in lignin may be a result from the oxidative combinatorial linking of the 4-hydroxyphenyl propanoid building blocks provided by nature. The aromatic portion of these building blocks is composed of 4-hydroxyphenyl, guaiacyl (4-hydroxy-3-methoxyphenyl), and syringyl (4-hydroxyl-3,5-dimethoxyphenyl) units. These units may be abbreviated as H, G, and S, respectively. The lignin itself may also vary in the ratio of these units depending on its source. A determination of these building blocks may establish what biobased products may be produced from the lignin.

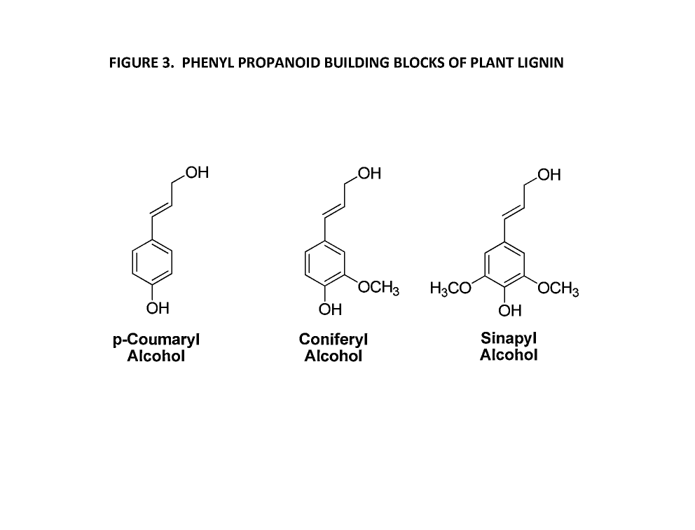

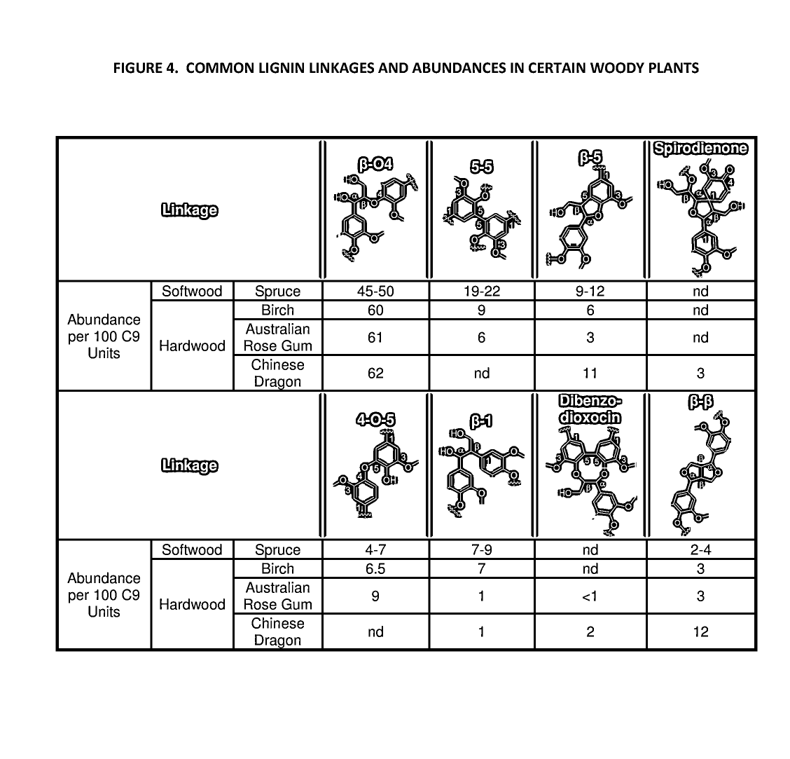

FIG. 1 describes a method for evaluation of lignin 16 which may comprise the steps of providing lignin from a source, wherein the composition of lignin is unknown. Next, the lignin is analyzed. After the lignin is analyzed, the composition of the lignin may be determined. Once the composition of the lignin is substantially determined, the user can then predict at least one biobased product that may be produced from the lignin. In order to provide at least one biobased product, two factors may be considered in determining the composition of the lignin 16. The first factor may be the determination of the H, G, and S building blocks. These building blocks are further described in FIG. 3. The second factor may be the determination of the linkages. These linkages are described further in FIG. 4. Together, these two factors may provide a determination of the lignin 16 composition which can predict at least one biobased product that may be produced.

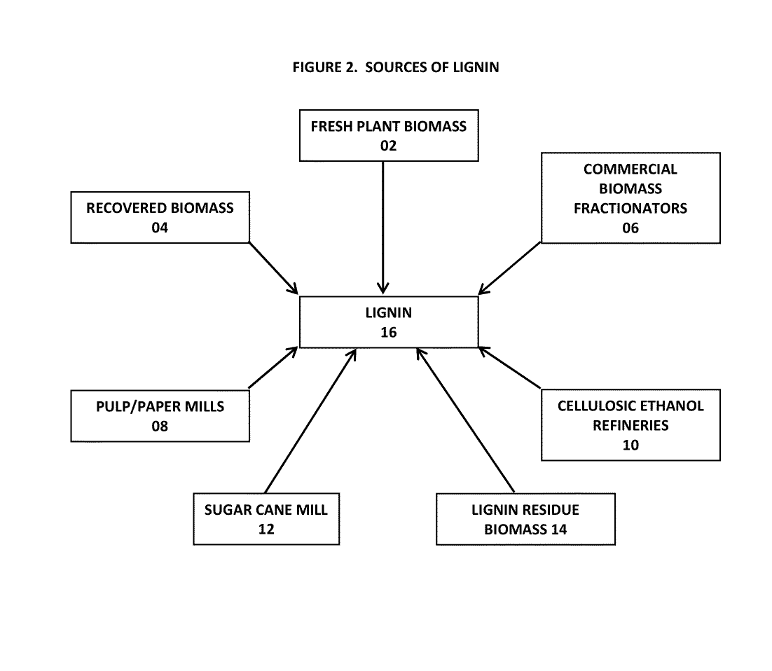

As lignin 16 is received from a source, its composition may be unknown. As further described below, such sources may include paper mills, cellulosic ethanol refineries, sugar cane mills, kraft pulp mills, sulfite pulp mills, soda pulp mills, or any plant, mill or refinery that produces biomass. These sources are described further in FIG. 2. Although the source may provide a guide as to what the range for the H, G, and S building blocks and linkages may be, the exact composition may be indefinite. In order to accurately predict at least one biobased product, a determination of the composition, namely the building blocks and/or linkages, within the lignin 16 may be completed to provide a determination of at least one biobased product that may be produced. For this determination of the composition of lignin 16, the method described herein utilizes the analysis of lignin 16 in order to determine at least one biobased product that may be produced. The method described herein may be both rapid and valuable for use in biobased product production operations.

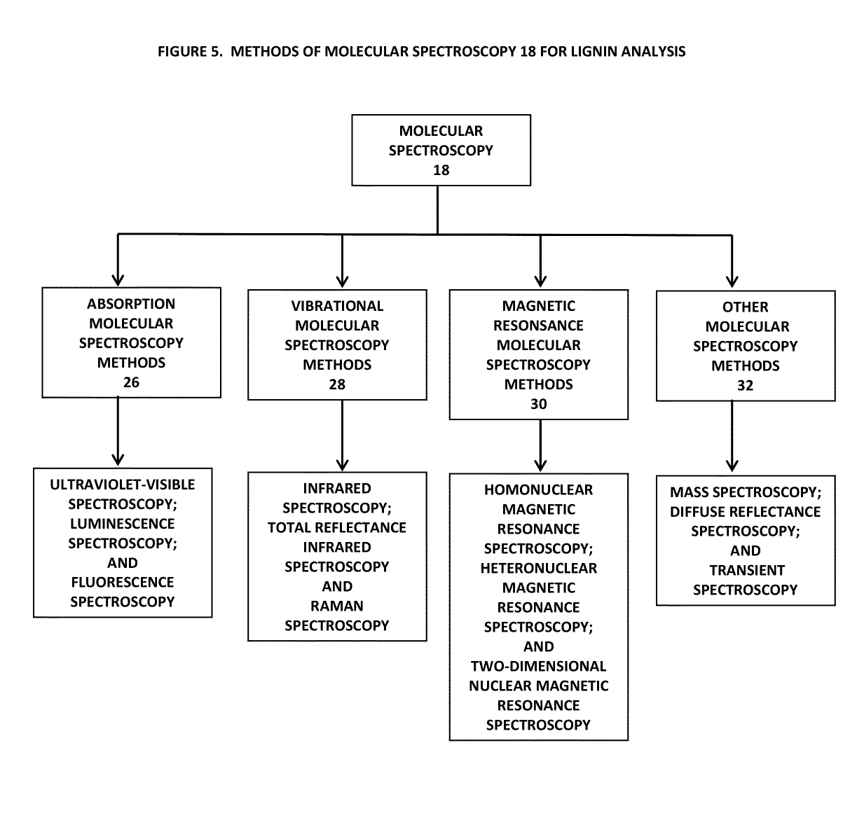

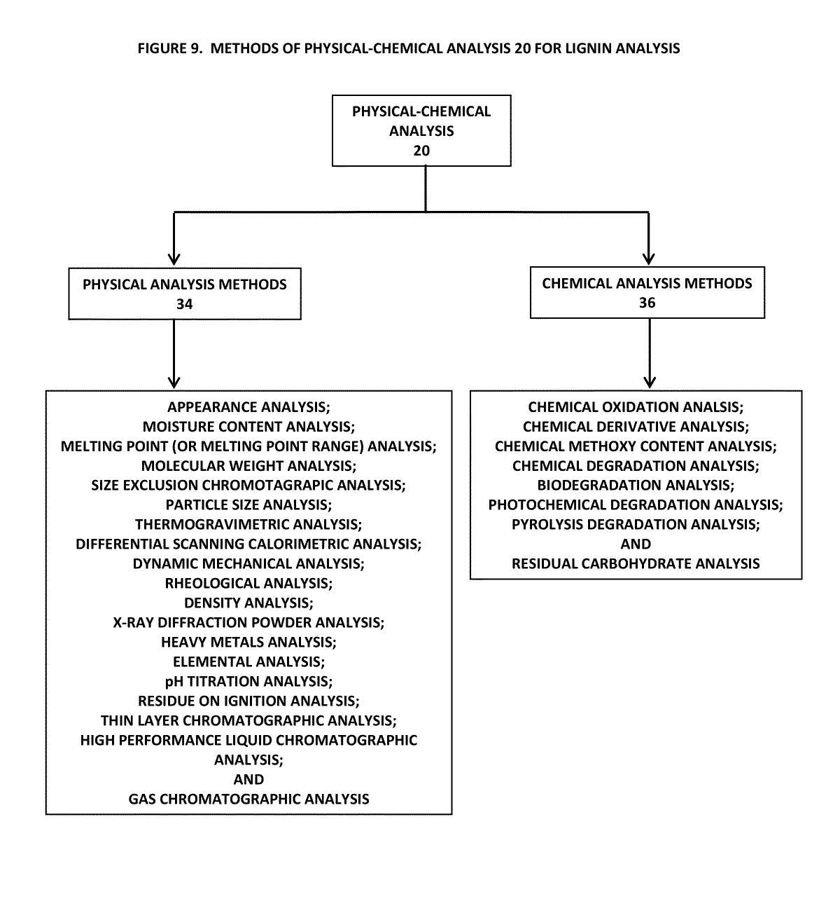

FIG. 1 indicates several possible pathways for lignin 16 analysis under which the method may be practiced. For the method described in FIG. 1, lignin 16 may be analyzed by molecular spectroscopy 18 and/or physical-chemical analysis 20. To note, either or both of the molecular spectroscopy 18 and/or physical-chemical analysis 20 methods may be used, and are described further in FIGS. 5 and 9, respectively. In one instance, lignin analysis may comprise at least one method of molecular spectroscopy 18 or at least one method of only physical-chemical analysis 20. In another instance, lignin analysis may comprise at least one method of both molecular spectroscopy 18 and physical-chemical analysis 20. If both the molecular spectroscopy 18 and physical-chemical analysis 20 are used within the lignin 16 analysis method, then any of the methods described above may be used in any order.

The analysis of lignin 16 by molecular spectroscopy 18 and/or physical-chemical analysis 20 may provide at least one experimental datum related to the molecular structure of lignin 16. The analysis of lignin 16 may also provide at least two experimental data related to the structure of lignin 16. The analysis of lignin 16 may further provide at least one experimental datum related to the p-coumaryl alcohol, coniferyl alcohol, and sinapyl alcohol content of lignin 16. Also, the analysis of lignin 16 may further provide at least two experimental data related to the p-coumaryl alcohol, coniferyl alcohol, and sinapyl alcohol content of the lignin 16, as described in FIG. 3.

After the experimental data or datum from at least one of molecular spectroscopy 18 and/or physical-chemical analysis 20 can be provided, a data analysis 22 step may then be completed. The method may allow lignin 16 to undergo data analysis 22 in order to allow for a predictive correlation 24 in determining at least one biobased product from the lignin 16. This data analysis step may include performing a statistical analysis of the experimental data or datum. This statistical analysis of experimental data may be provided by at least one computational method of manual computation, spreadsheet computation, website computation, and/or statistical software computation. Such data from molecular spectroscopy 18 and/or physical-chemical analysis 20 may be processed either manually or electronically. The statistical analysis of experimental data can be conducted on a calculator or a computer.

The statistical analysis described above may further include regression analysis, wherein the regression analysis of the experimental data may be provided by at least one regression analysis method of linear regression analysis and/or multivariate regression analysis. The regression analysis of experimental data may also be provided by linear least squares regression analysis. Regression analysis of the experimental data may be comprised of at least one computational method of manual computation, spreadsheet computation, website computation, and/or statistical software computation. Regression analysis of the experimental data may be conducted on a calculator or a computer and processed either manually or electronically.

In the process described herein, a predictive correlation 24 may be provided from the method described, wherein data analysis 22 of experimental data obtained by analyzing the lignin 16 may provide the ability to predict the product identity of at least one biobased product that may be produced from the lignin 16. Furthermore, the predictive correlation 24 may provide the ability to predict the product identities of at least two biobased products that may be produced from the lignin 16. In the ability to forecast the product identity of at least one biobased product produced from the lignin 16, the step of using the determination of the composition to predict at least one biobased product produced from the lignin may have an accuracy of about 70% to about 100%. Additionally, the step of using the determination of the composition to predict at least one biobased product produced from the lignin may have an accuracy of about 85% to about 100%. Also in the process described herein, a predictive correlation 24 step may be provided from the lignin 16 analysis method, wherein data analysis 22 of experimental data obtained by analyzing the lignin 16 may provide the ability to predict the product yield of at least one biobased product produced from the lignin 16. Also, the predictive correlation 24 step may provide the ability to predict the product yields of at least two biobased products that may be produced from the lignin 16. In the ability to predict the product yield of at least one biobased product produced from the lignin 16, the step of determining the composition of the lignin may provide the prediction of a product yield of at least one biobased product produced from the lignin with an accuracy of about 70% to about 100%. Additionally, the step of determining the composition of the lignin may provide the prediction of a product yield of at least one biobased product produced from the lignin with an accuracy of about 85% to about 100%.

In the method of analysis of lignin 16 for the process described herein, the analysis may require less than about 2 hours to perform. The analysis of lignin 16 may also require less than about 30 minutes to perform. The method of analysis of lignin 16 may be automated as well.

FIG. 2 provides a schematic overview where lignin 16 may be provided from various sources. The sources for the lignin 16 may include fresh plant biomass 2, recovered biomass 4, commercial biomass fractionators 6, pulp and paper mills 8, cellulosic ethanol refineries 10, sugar cane mills 12, and/or lignin residue biomass 14. These sources may provide particular ranges for what the H, G, and S building blocks may be, and a lignin 16 source may be chosen to provide at least one biobased product to be produced. If the lignin 16 source may not be chosen, then the determination of the H, G, and S building blocks of the lignin 16 source may assist in determining at least one biobased product to be produced.

Because of its make-up, lignin can be a source of aromatic chemicals outside of the conventional sources of petroleum and coal. Lignin may be obtained from wood and/or agricultural sources as fresh biomass. This wood and/or agricultural lignin may be waste lignin or recovered lignin from these sources. Lignin can also be obtained from multiple sources that utilize plant material, including pulp and paper mills and the sugar cane milling industries. It is also a major by-product in the cellulosic biomass-to-ethanol process. Often, these sources of lignin may be considered waste products where there may be an associated cost to dispose of the lignin instead of alternative methods where this lignin may provide value-added products.

Another source of lignin is the black liquor produced from kraft pulp mills. In the kraft pulping process, lignin-rich black liquor is burnt in a recovery boiler to recover the spent alkali and to generate heat and power for mill operations. Some of the lignin in black liquor may be precipitated and used for value-added applications, especially since a production bottleneck may exist from the thermal capacity of the recovery boiler.

A description of producing lignin from biomass may be found in applications commonly owned by the assignee of the instant application, including A METHOD FOR PRODUCING BIOBASED CHEMICALS FROM PLANT BIOMASS (U.S. application Ser. No. 13/292,222 filed Nov. 9, 2011), A METHOD FOR PRODUCING BIOBASED CHEMICALS FROM WOODY BIOMASS (U.S. application Ser. No. 13/292,437 filed Nov. 9, 2011), A METHOD FOR PRODUCING BIOBASED CHEMICALS FROM AGRICULTURAL BIOMASS (U.S. application Ser. No. 13/292,531 filed Nov. 9, 2011), and A METHOD FOR PRODUCING BIOBASED CHEMICALS FROM CULTIVATED PLANT BIOMASS (U.S. application Ser. No. 13/292,632 filed Nov. 9, 2011), A METHOD FOR PRODUCING BIOBASED CHEMICALS FROM PLANT LIGNIN (U.S. Application No. 61/608,936 filed Mar. 9, 2012), A METHOD FOR PRODUCING BIOBASED CHEMICALS FROM PLANT LIGNIN (U.S. application Ser. No. 13/453,422 filed Apr. 23, 2012), A METHOD OF PRODUCING BIOBASED CHEMICALS FROM PLANT BIOMASS (PCT application U.S. Ser. No. 12/62942 filed Nov. 1, 2012), which are incorporated herein by reference. Further, applications commonly owned by the assignee of the instant application may describe selective production of one or more biobased products from lignin, including A METHOD FOR SELECTIVE PRODUCTION OF BIOBASED CHEMICALS AND BIOFUELS FROM PLANT LIGNIN (U.S. application Ser. No. 13/470,398 filed on May 14, 2012) and a MECHANISM FOR PRODUCTION OF BIOBASED PRODUCTS FROM PLANT LIGNIN (U.S. Application No. 61/646,475 filed on May 14, 2012), which are also incorporated herein by reference.

The applications listed above may provide some methods for producing one or more biobased chemicals from lignin. After the lignin 16 is obtained using these methods or through other means, it may be analyzed for predicting one or more biobased products obtained from lignin. These methods for the evaluation of lignin feedstock are described herein. These methods may also provide for a selective production of the biobased chemicals based on the analysis of the lignin provided.

Lignin 16 may be the most abundant source of aromatic chemicals outside of crude oil and coal. Lignin 16 can be used in developing technologies that transform various sources of biomass and lignin 16 waste into value-added aromatic chemicals. The sources of lignin 16 may include at least one biomass of plant biomass, woody plant biomass, agricultural plant biomass, and/or cultivated plant biomass. The sources of lignin 16 may include fresh plant biomass 2, recovered biomass 4, commercial biomass fractionators 6, pulp and paper mills 8, cellulosic ethanol refineries 10, sugar cane mills 12, and/or lignin residue biomass 14. Although these sources of lignin 16 can be used, these sources of lignin 16 are not limited to only those listed herein. No matter the origin of the lignin 16, any different sources of lignin 16 may be used in the conversion of lignin 16 to one or more biobased products. This conversion process may provide one or more biobased products that can include biobased chemicals, biofuels, biobased materials, and/or lignin residues.

Lignin 16 may be a structurally complex, polymeric substance made up of 4-hydroxyphenyl propanoid building blocks containing 4-hydroxyphenyl (abbreviated as H), guaiacyl (4-hydroxy-3-methoxyphenyl) (abbreviated as G), and syringyl (4-hydroxy-3,5-dimethoxyphenyll) units (abbreviated as S). The lignin 16 building blocks are described further in FIG. 3. The abundance of each of these units within the lignin 16 may change somewhat between individual plant species for woody lignin, namely lignin content for hardwoods and softwoods, as well as for agricultural sources and both cultivated and uncultivated plants. This difference in the units based on the species for the lignin 16 may control, or at least predict, the amounts and types of chemical products that may be produced within the conversion of lignin 16 to one or more biobased products.

To begin the conversion process, fresh plant biomass 2 may be utilized as a lignin source. Fresh plant biomass 2 may be considered to be biomass from agricultural plants, woody plants, and/or other plant biomass sources. Fresh plant biomass 2 may also include cultivated plant biomass. Fresh plant biomass 2 may be used where it may be grown specifically for this application, which may include, but is not limited to, switchgrass, miscanthus, hybrid eucalyptus trees, and hybrid poplar trees. Some fresh plant biomass 2 not specifically grown for this application may include agricultural or tree harvesting surplus, and natural grasses and forest trees. Where fresh plant biomass 2 is used, the lignin 16 can be separated from the other components like cellulose, hemicellulose, and other extractives. After the lignin 16 is separated, it may be added to the conversion process.

Sources of recovered biomass 4 may include several biomass waste products. The recovered biomass 4 can include woody biomass like wood chips, sawdust, and/or recovered wood, and/or agricultural plant biomass like wheat straw, rice straw corn stover and/or other agricultural products typically left to rot in the field. Additionally, other plant biomass may also include lawn and tree maintenance byproducts. Another potential source of lignin 16 from recovered biomass 4 may include sugar cane milling. Sugar cane milling may provide lignin 16 since bagasse, or sugarcane waste fiber, can be generated. Bagasse is the name given to the discarded husks of the sugarcane plant after they have been pressed to extract the juices which are refined to make sugar. This agricultural waste can be very plentiful and may otherwise be burnt or discarded in the sugar cane milling process. Recovered biomass 4 may also include other waste products, including at least one waste lignin of sulfite pulping mill waste lignin, kraft pulping mill waste lignin, soda pulping mill waste lignin, and/or sugar cane mill waste lignin. Both the fresh plant biomass 2 and the recovered biomass 4 may be treated to provide lignin 16 using any of the conversion methods described in assignees applications incorporated within the present application.

Another source of lignin 16 may be commercial biomass fractionators 6. These commercial biomass fractionators 6 may use a chemical, thermal and/or mechanical processor which directly inputs raw biomass such as fresh plant biomass 2, woodchips and crop waste and produces multiple component streams, which may include sugars, cellulose, hemicellulose, and lignin 16. One example of a commercial biomass fractionator 6 may be Vertichem Corporation. Some of these component streams may include lignin 16 streams to produce useful products such as aryl aldehydes, aryl carboxylic acids, aryl esters, aryl ketones, aryl alcohols, aliphatic carboxylic acids, phenols, alkyl phenols, alkenylphenols, benzene, toluene, xylene (collectively, benzene, toluene, and xylene are often referred to as “BTX”), mesitylenes, biaryls, aryl alkanes, aryl alkenes, alkanes, alkenes, cycloalkanes, cycloalkenes, alkyl esters, performance chemicals, biofuels, and/or biomaterials. Within the conversion process, the biomass may be treated to yield a highly pure cellulose fraction. Several different methods may be used for the separation, including pH, temperature, and pressure adjustments. A reaction involving enzymes may also be used. Other methods of fractionation may include chemical, mechanical, and biological methods. For instance, the biomass fractionator may separate the cellulose out by hot water treatments, hot acidic treatments, hot alkaline treatments, and/or an alkaline oxidation step. Although the commercial biomass fractionators 6 may provide useful biobased products, they may also produce or leave behind other solids comprising of lignin 16. Instead of becoming a waste product, these lignin 16 solids may be used within the conversion process(es) referenced above.

Pulp and paper mills 8 may also contribute to the lignin 16 from kraft pulping, sulfite pulping, and soda pulping. Lignin 16 can be removed during paper processing in a pulp and paper mills 8, where it is typically viewed as an undesirable component of biomass that requires both energy and chemicals to remove it during the pulping operation. These pulp and paper mills 8 may generally recover the lignin 16 as a by-product of the pulping process and may use it as boiler fuel. This removal of lignin 16 may be done by a chemical removal, with or without mechanical means. Some chemical methods of lignin 16 removal from pulp and paper mills 8 may be kraft pulping, sulfite pulping, and soda pulping.

The more dominant chemical pulping technique employed can be kraft processing, which employs high pHs by using considerable amounts of aqueous sodium hydroxide and sodium sulfide at high temperatures to degrade cellulosic biomass into cellulose, hemicellulose, and lignin 16 in a stepwise process. In the kraft process, black liquor can be burnt in a recovery boiler to recover the spent alkali and to generate heat and power for mill operations. However, some of the lignin 16 in black liquor can be precipitated and used for value-added applications where these exist. This conversion to value-added applications may be particularly attractive for a kraft pulping mill where a production bottleneck exists due to the thermal capacity of the recovery boiler. This conversion process may also provide kraft lignin.

The sulfite processing yielding lignosulfonates can also be relatively common in the pulp and paper industry. The sulfite process may be conducted between about pH 2 to about pH 12 using sulfite with a counterion. This counterion may be either calcium or magnesium. The product may be soluble in water as well as some highly polar organics and amines.

The soda pulp mill may also provide another chemical pulping process where caustic soda can be used to produce pulp. Although it is an old method, it can be effective in separating pulp from wood and grasses.

Another source of lignin 16 may also be cellulosic ethanol refineries 10. With the cellulosic ethanol refineries 10, they may produce lignin 16 and other by-products in the cellulosic biomass-to-ethanol process, which can also be used to produce energy required for the ethanol production process. Cellulosic ethanol refineries 10 produce ethanol fuel. The cellulosic ethanol can be made from plant materials like switchgrass, miscanthus, wheat stalks, corn stover, and woody biomass.

Cellulosic ethanol refineries 10 may use the OrganoSolv™ process or the Alcell® process to obtain lignin 16. OrganoSolv™ lignin may be obtained by treatment of fresh plant biomass 2 or bagasse. The fibrous residue that remains after plant material may be treated with various organic solvents. The OrganoSolv™ process may produce separate streams of cellulose, hemicelluloses, and lignin 16. It can be considered environmentally friendly because it may not use the sulfides, sulfites, and harsh conditions used in the kraft or lignosulfonate pulping processes, but it can have a higher cost because of the solvent recovery in this process. Some processes that may be used to separate the biomass to obtain lignin 16 may include any of the methods described in U.S. utility applications commonly owned by the assignee of the instant application from Applicant, which are incorporated herein. Another process to obtain lignin 16 that may be used at cellulosic ethanol refineries 10 may include acidic hydrolysis and/or enzymatic reactions. Typically, the lignin 16 recovered from the cellulosic ethanol refineries 10 may be used as boiler fuel. Additionally, the lignin 16 recovered from the cellulosic ethanol refineries 10 may undergo a pretreatment prior to entry into the process(es) described herein. The purpose of this lignin pretreatment may be to remove unwanted impurities from the lignin 16 and may include a series of steps to further separate lignin 16 from the other components of biomass such as cellulose and hemicellulose as well as the fats, oils, resins, pitches, waxes, other extractables that may be present in the biomass, or the salts, enzymes, and cellular debris that may be contaminating the lignin from biomass processing.

Besides the other sources for lignin 16, sugar cane mills 12 may also provide lignin 16 used in the process described herein. Sugar cane mill 12 biomass can include bagasse by-product from sugar cane processing to produce sugar. Bagasse, the fibrous matter that remains after sugarcane or sorghum stalks are crushed to extract their juice, may often be used as a primary fuel source for sugar mills. The bagasse may be burned, producing sufficient heat energy to supply all the needs of typical sugar cane mills 12. However, there may be an excess of bagasse when the energy supply to the sugar cane mills 12 has been provided.

Yet another source of lignin 16 may be lignin residue biomass 14. Lignin residue biomass 14 may include lignin residue caustic solution by-product or recovered solid depolymerized lignin residue from tiered biobased chemical, biofuel, and/or biomaterial production, as described in applications commonly owned by the assignee of the instant application, which are incorporated herein.

Although several sources for lignin are presented herein, those sources for lignin are not limited to those listed. Any lignin 16 provided may be used within any of the conversion processes described above to create one or more value-added products. Producing these chemicals may provide a reduction in the costs associated with waste disposal of lignin 16 and a means to generate income from biobased product production. Besides waste product sources of lignin 16 for the recovered biomass 4, lignin 16 waste from the lignin 16 processing may also provide a source for producing energy. This waste may include recovered plant biomass waste lignin, kraft pulp mill waste lignin, sulfite pulp mill waste lignin, soda pulp mill waste lignin, cellulosic ethanol refinery waste lignin, sugar cane mill waste lignin, and commercial biomass fractionators waste lignin. In this reduction of waste for the process described herein, the waste product of the lignin biomass may be less than 30% of the lignin weight. It may also be less than 20% of the lignin weight. It may also be less than 10% of the lignin weight. These waste products, although reduced, may be used to produce energy which utilizes the waste product, providing value to the process. This energy production may be heat and/or power.

FIG. 3 provides some of the chemical building blocks of lignin 16 that may be used to determine at least one biobased product that may be produced from the lignin 16 provided. Lignin 16 constitutes one of the three major components of lignocellulosic biomass, of which the other two major components are cellulose and hemicellulose. The polymeric structure of lignin 16 can be very complex and a complete structure elucidation of any single lignin is still unknown. The building block compositions of lignin, the extent of polymerization, and the abundance of lignin can vary from plant species to plant species. The H:G:S ratio of lignin may then be used advantageously in the selection of a suitable feedstock (i.e., a specific plant species, and/or a biomass treatment method, and/or blend of different lignin types) for the production of a specific biobased product and/or to achieve a certain ratio of specific biobased products. This composition may therefore provide control of the composition of one or more biobased product from lignin 16. The abundance of lignin in plants generally may decrease from softwoods to hardwoods, and also may decrease from hardwoods to grasses. Moreover, lignin structure may be impacted by the treatment process used to separate lignin from the other components of biomass.

Lignin 16 may be an amorphous polymer made up of three phenyl propanoid building blocks shown in FIG. 3. These building blocks may differ in the degree of oxygen substitution on the phenyl ring, and/or in the degree of methoxy substitution on the phenyl ring. In nature, lignin may impart strength and rigidity to the plant by extensive cross linking with polymeric hemicellulose and/or cellulose.

Most plant lignin 16 types may be comprised of all three building blocks shown in FIG. 3. Depending on the species of plant, the ratio of these three building blocks may vary. The composition of lignin may frequently be stated in terms of its 4-hydroxyphenyl (H), guaiacyl (G), and sinapyl (S) content. These aromatic systems may correspond, respectively, to the p-coumaryl alcohol, coniferyl alcohol, and sinapyl alcohol building blocks of lignin. First, the p-coumaryl alcohol building block may correspond to the p-hydroxyphenyl (H) make-up of lignin. Agricultural and grassy plants like wheat straw, rice straw, switchgrass and corn stover may tend to have the highest H contents. Two H-derived oxidation products of lignin may include 4-hydroxybenzaldehyde and 4-hydroxybenzoic acid. Second, the coniferyl alcohol building block may correspond to the guaiacyl (G) make-up of lignin. Softwoods like spruce, balsam and pine, in general, may tend to have the highest G content, often in excess of about 80% of the plant lignin. Two G-derived oxidation products of lignin may include vanillin and vanillic acid. Third, the sinapyl alcohol building block may correspond to the sinapyl (S) make-up of lignin. Typically, hardwoods may have high S contents, which may be over 50% where the balance may be comprised predominantly of G. Some examples of hardwoods may include willow, birch, maple, aspen and oak. Two S-derived products may include syringaldehyde and syringic acid. The values shown below in Table A may provide normalized H:G:S ratios found in certain lignin 16 by selective α-β bond cleavage through lignin oxidative depolymerization of the C9 phenyl propanoid building blocks (the α-β linkage is shown further in FIG. 4):

TABLE A
Normalized H:G:S Ratios of Various Plant Lignins

| Entry | Group | Species | % H | % G | % S |
|---|---|---|---|---|---|
| 1 | Hardwood | Eucalyptus globulus | NR | 1.00 | 4.04 |
| 2 | | Red Oak | NR | 1.00 | 2.43 |
| 3 | | Cottonwood | NR | 1.00 | 1.53 |
| 4 | | Sweet Gum | NR | 1.00 | 1.44 |
| 5 | | Acacia | NR | 1.00 | 1.04 |
| 6 | | White Birch | NR | 1.00 | 2.57 |
| 7 | | Silver Birch | NR | 1.00 | 3.76 |
| 8 | | Red Alder | NR | 1.00 | 1.22 |
| 9 | | Red Maple | NR | 1.00 | 1.4 |
| 10 | | Salix integra | NR | 1.00 | 1.94 |
| 11 | | Poplar (Cradon, Arlington, WI) | NR | 1.00 | 1.44 |
| 12 | | Poplar (W79, Wallula, WA) | NR | 1.00 | 1.03 |
| 13 | Softwood | Mixed Softwood Kraft | 0.09 | 1.00 | 0.08 |
| 14 | | Black Spruce, milled only | 0.08 | 1.00 | 0.11 |
| 15 | | Black Spruce, alkaline treatment | 0.08 | 1.00 | 0.13 |
| 16 | | White Spruce | NR | 1.00 | 0.00 |
| 17 | | Norway Spruce | NR | 1.00 | 0.00 |
| 18 | | Red Pine | NR | 1.00 | 0.00 |
| 19 | | Monterey Pine | NR | 1.00 | 0.00 |
| 20 | | Loblolly Pine | NR | 1.00 | 0.00 |
| 21 | Agri- | Wheat Straw, milled only | 1.33 | 1.00 | 0.18 |
| 22 | cultural, | Wheat Straw, alkaline treatment | 0.43 | 1.00 | 0.16 |
| 23 | Grasses | Wheat Straw, acid treatment | 0.98 | 1.99 | 0.20 |
| 24 | | Rice Straw | 0.72 | 1.00 | 0.46 |
| 25 | | Corn Stover | 1.30 | 1.00 | 0.94 |
| 26 | | Switchgrass | NR | 1.00 | 0.27 |

* NR = Not reported

Based on the lignin provided, the product distribution may parallel the H:G:S ratio. Selection of the lignin source may therefore allow for the prediction of a certain product ratio. For example, if high levels of G-derived products are desired, then a lignin composition of high G content may be preferred. These high-level of G-derived products may be obtained from either a specific lignin, which may include a specific plant species or biomass pretreatment method, that may provide a lignin of high G content and/or a blend of different lignin forms such that the blend may have the desired G content.

Besides the different H:G:S ratios from the different species, there may also be a difference in the H:G:S ratio after the biomass pretreatment method, even within the same plant species (see Table A wheat straw entries 21-23). For these different plant species and also lignin obtained from different biomass pretreatment methods, many different chemical linkages may occur between the three building blocks. Some of these common linkages may be seen in FIG. 4.

No matter the lignin 16 source or type of treatment, the polymeric structure of lignin may be complex and a complete structure for any single lignin is unknown. Further, samples of lignin obtained from a single lignin source may also differ in its polymeric structure, providing variable building block compositions even within the same sample.

FIG. 4 provides some common linkages and abundances in certain woody softwood and hardwood plants. The determination of these linkages may also provide a prediction as to at least one biobased product that may be produced. For the common linkages provided, there may be an abundance of a particular linkage within some specific species, as provided in FIG. 4. Although numbers have been provided within the figure, these numbers may vary due to other factors which may include but are not limited to lignin treatment, growth rate of the plant, environmental factors, region where growth of the plant occurs, and/or genetic differences of the plant.

For the lignin linkages of these softwoods and hardwoods, there may be at least 8 different linkages which may be commonly found. These linkages may include: β-O-4, 5-5, β-5, 4-O-5, β-1, β-β, spirodienone, and dibenzodioxocin. The abundance of these linkages may be measured by their prevalence per 100 C9 units.

The predominant linkage structure in lignin may be a β-O-4 linkage. This linkage may account for about 45% to about 60% or more of all linkages in woody lignin. In other types of plant lignin, this number may vary. For example, this linkage may reach about 80% or more in corn stover lignin. The linkage designation of β-O-4 can refer to a carbon-oxygen bond between the β-carbon, which is the central carbon of the propyl side chain of one building block, with the 4-hydroxy group on the phenyl ring of a second lignin phenyl propanoid building block. A β-O-4 linkage may occur between and among H, G, and S building blocks.

Another notable linkage may be the 5-5 linkage. The 5-5 linkage type can refer to a carbon-carbon bond between C-5 positions of two phenyl rings of two different phenyl propanoid building blocks. This linkage type may be common in some softwoods, but may not be as prevalent in some hardwoods. A 5-5 linkage may occur between and among H and G building blocks. An S building block may not enter into a 5-5 linkage because the C-5 position of S is occupied by a methoxy group and prevents this linkage.

The β-5 linkage may also be found in both softwoods and hardwoods. The β-5 linkage can refer to a carbon-carbon bond between the β-carbon position of one building block and C-5 position of the phenyl ring of a second building block. A β-5 linkage may occur between and among H, G, and S building blocks, although the building block comprising the C-5 linkage position may not be S because the C-5 position of S is occupied by a methoxy group and prevents this linkage.

The 4-O-5 linkage may be another linkage found in certain woody plants. The 4-O-5 linkage can refer to an ether linkage, which can comprise an oxygen-carbon bond, between a 4-hydroxyphenyl group of one building block with the C-5 position on a phenyl ring of a second building block. A 4-O-5 linkage may occur between and among H, G, and S building blocks, although the building block comprising the C-5 linkage position may not be S because the C-5 position of S is occupied by a methoxy group and prevents this linkage.

Another linkage may also include a β-1 linkage. The β-1 linkage can occur through a carbon-carbon bond between the β-carbon of one building block and position 1 of another phenyl ring. A β-1 linkage may occur between and among H, G, and S building blocks.

Yet another linkage may include a β-β linkage. A β-β linkage can be a carbon-carbon bond between the βpositions of two building blocks, generally leading to a fused bis-furan system. This linkage may occur between any of the three building blocks. This linkage may occur between and among H, G, and S building blocks.

Some other linkages, although not as common or prevalent as some of the aforementioned linkages, may be the spirodienone and dibenzodioxocin linkages. The spirodienone and dibenzodioxocin linkages can be multifunctional linkages. These linkage types, however, may not be seen across all lignin types. The spirodienone linkage may occur with any of the three building blocks, whereas the dibenzodioxocin linkage may only occur with p-coumaryl alcohol and coniferyl alcohol building blocks since the 5-methoxy group of sinapyl alcohol prevents its formation. The spirodienone linkage may occur between and among any of the three building blocks, whereas the biobenzodioxocin linkage may only occur with H or G because the C-5 position of S is occupied by a methoxy group and prevents this linkage.

To note, those linkages in FIG. 4 may be commonly found linkages in certain woody plants. They are, however, not an exhaustive list of all linkages found. Further, not all linkages can be seen in every lignin type, and the ratio of these linkages may change between different plant species and between different lignin pretreatments even within the same plant species.

FIG. 5 provides various methods of molecular spectroscopy 18 that may be used for lignin 16 analysis. These methods of molecular spectroscopy 18 may comprise at least one molecular spectroscopy 18 method of absorption molecular spectroscopy methods 26, vibrational molecular spectroscopy methods 28, magnetic resonance molecular spectroscopy methods 30, and/or other molecular spectroscopy methods 32. Additionally, the methods of molecular spectroscopy 18 may comprise at least two molecular spectroscopy methods of absorption molecular spectroscopy methods 26, vibrational molecular spectroscopy methods 28, magnetic resonance molecular spectroscopy methods 30, and/or other molecular spectroscopy methods 32. For the process described herein, at least one molecular spectroscopy 18 method of absorption molecular spectroscopy methods 26, vibrational molecular spectroscopy methods 28, magnetic resonance molecular spectroscopy methods 30, and/or other molecular spectroscopy methods 32 may be conducted along with at least one method of physical-chemical analysis 20.

Absorption molecular spectroscopy methods 26 can be molecular spectroscopy 18 methods based on absorption or emission of a photon of light by a molecule and comprises at least one method of ultraviolet-visible spectroscopy, luminescence spectroscopy, and/or fluorescence spectroscopy. Ultraviolet-visible spectroscopy may use light in the visible and adjacent (near-UV and near-infrared) ranges. The absorption or reflectance in the visible range can directly affect the perceived color of the chemicals involved. In this region of the electromagnetic spectrum, molecules can undergo electronic transitions. This technique may be complementary to fluorescence spectroscopy, in that fluorescence spectroscopy may deal with transitions from the excited state to the ground state, while ultraviolet-visible spectroscopy can measure transitions from the ground state to the excited state. Luminescence can be a light emission which represents an excess over the thermal radiation and lasts for a time exceeding the period of electromagnetic oscillation. Luminescent spectroscopy may be performed using the intrinsic luminescence of materials under observation, or when special markers called luminophors may be added when the material itself does not demonstrate luminescent properties. Fluorescence spectroscopy may be a “fast” photoluminescence.

Vibrational molecular spectroscopy methods 28 may also be molecular spectroscopy 18 methods. Vibrational molecular spectroscopy methods 28 may be based on bond vibrational, rotational-vibrational, or stretching motions induced in a molecule by an externally applied energy. These methods may comprise at least one method of infrared spectroscopy, total reflectance infrared spectroscopy, and/or Raman spectroscopy. These methods may further comprise Fourier transform techniques thereof.

Infrared spectroscopy can involve the infrared region of the electromagnetic spectrum, which is light of a longer wavelength and lower frequency than that of visible light. The infrared portion of the electromagnetic spectrum can usually be divided into three regions; the near-, mid-, and far-infrared regions, named for their relation to the visible spectrum. The higher-energy near-infrared, approximately about 14000 cm⁻¹ to about 4000 cm⁻¹ (about 0.8 μm to about 2.5 μm wavelength), may excite overtone or harmonic vibrations. The mid-infrared, approximately about 4000 cm⁻¹ to about 400 cm⁻¹ (about 2.5 μm to about 25 μm), may be used to study the fundamental vibrations and associated rotational-vibrational structure. The far-infrared, approximately about 400 cm⁻¹ to about 10 cm⁻¹ (about 25 μm to about 1000 μm), lying adjacent to the microwave region, can have low energy and may be used for rotational spectroscopy. The infrared spectrum of a sample may be recorded by passing a beam of infrared light through the sample. When the frequency of the infrared is similar to the vibrational frequency of a bond in the molecule, absorption may occur. Examination of the transmitted light may reveal how much energy was absorbed at each frequency and/or wavelength. This may be achieved by scanning the wavelength range using a monochromator. Alternatively, the whole wavelength range may be measured at once using a Fourier transform instrument, and then a transmittance or absorbance spectrum may be generated using a dedicated procedure. Analysis of the position, shape and intensity of peaks in this spectrum may reveal details about the molecular structure of the sample. A variety of methods exist for analyzing liquids and solids by infrared spectroscopy. These methods may include coating a liquid or solid mull onto salt plates, or salt presses for solids. An alternative method of obtaining an infrared spectrum of a sample is total reflectance (often referred to at attenuated total reflectance infrared spectroscopy). Total reflectance infrared spectroscopy may offer the advantage of a substantially shorter sample preparation and analysis time because the sample may be coated onto a reflectance crystal which is comprised of an infrared transparent material with a high refractive index and polished surfaced. The infrared beam then enters the side of the crystal at an angle such that the infrared light may be reflected at the sample crystal interface. In the spectral regions where the sample absorbs energy, the light wave may be attenuated. The attenuated light exiting the crystal is then sent to a detector to produce an infrared spectrum of the sample.

Raman spectroscopy is another spectroscopic technique used to study vibrational, rotational, and other low-frequency modes in a molecule. Raman spectroscopy relies on inelastic scattering, or Raman scattering, of monochromatic light, usually from a laser in the visible, near infrared, or near ultraviolet range. The laser light interacts with molecular vibrations, phonons or other excitations in the system, resulting in the energy of the laser photons being shifted up or down. The shift in energy can give information about the vibrational modes in the system. Infrared spectroscopy yields similar, but complementary, information to Raman spectroscopy. Typically, a sample is illuminated with a laser beam. Light from the illuminated spot is collected with a lens and sent through a monochromator. As described above for infrared spectroscopy, Raman spectroscopy may also be conducted in a total reflection mode.

Magnetic resonance molecular spectroscopy methods 30 can be molecular spectroscopy 18 methods based on radiofrequency-induced spin flips of nuclei in the presence of an applied magnetic field. When placed in a magnetic field, nuclear magnetic resonance active nuclei (such as ¹H or ¹³C) may absorb electromagnetic radiation at a frequency characteristic of the isotope. The resonant frequency, energy of the absorption, and the intensity of the signal may be proportional to the strength of the magnetic field. Magnetic resonance molecular spectroscopy 30 can be a powerful technique that may provide detailed information on the topology, dynamics and three-dimensional structure of molecules in solution and the solid state. Its methods may comprise at least one method of homonuclear magnetic resonance spectroscopy, heteronuclear magnetic resonance spectroscopy, and/or two-dimensional nuclear magnetic resonance spectroscopy. Nuclear magnetic resonance spectroscopies of importance to lignin analysis may include that of at least one of proton (¹H) and/or carbon (¹³C) nuclei. Phosphorylation of lignin by chemical techniques may provide for lignin analysis by phosphorous (³¹P) nuclear magnetic resonance spectroscopy; although this may be a more laborious analysis method. Heteronuclear magnetic resonance spectroscopy may provide structural correlation information between two proximate nuclei such as ¹H and ¹³C. Heteronuclear magnetic resonance spectroscopy may be conducted as one-dimensional or two-dimensional modes. Two-dimensional nuclear magnetic resonance spectroscopy may further provide for intermolecular and through space proximity assignments of nuclei. These methods may further comprise Fourier transform techniques thereof.

Other molecular spectroscopy methods 32 may comprise a collection of molecular spectroscopy 18 methods that find specific applications in molecular structure assignments. The molecular spectroscopy methods 32 can include mass spectroscopy, diffuse reflectance spectroscopy, and/or transient spectroscopy. In one of the methods, mass spectroscopy is an analytical technique that may measure the mass-to-charge ratio of charged particles. It can be used for determining masses of particles, for determining the elemental composition of a sample or molecule, and for elucidating the chemical structures of molecules. Mass spectroscopy may work by ionizing chemical compounds to generate charged molecules or molecule fragments and measuring their mass-to-charge ratios. In another method, diffuse reflectance spectroscopy may be used for measurement of fine particles and powders, as well as a rough surface. Sampling may be fast and simple because little or no sample preparation is required. With powdered lignin, diffuse reflectance spectroscopy may simplify sample preparation. Diffuse reflectance spectroscopy may be applied as a technique to vibrational molecular spectroscopy methods 28 or absorption molecular spectroscopy methods 26 (i.e., reflectance infrared spectroscopy). In yet another method, transient spectroscopy may encompass a powerful set of techniques for probing and characterizing the electronic and structural properties of short-lived excited states (transient states) of photochemically/photophysically relevant molecules. These states may be accessed upon absorption of photons and essentially represent higher energy forms of the molecule, differing from the lowest energy ground state in the distribution of electrons and/or nuclear geometry.

For the method described herein, molecular spectroscopy 18 may comprise at least one method of infrared spectroscopy, total reflectance infrared spectroscopy, Raman spectroscopy, and/or total reflectance Raman spectroscopy. Also for the method described herein, molecular spectroscopy 18 may comprise at least one method of homonuclear magnetic resonance spectroscopy and/or heteronuclear magnetic resonance spectroscopy. With the use of infrared spectroscopy, total reflectance infrared spectroscopy, Raman spectroscopy, and/or total reflectance Raman spectroscopy for lignin 16 analysis, the limited solubility of the lignin 16 may not affect the analysis, since the analysis may be performed on the lignin 16 powder as a mull, a salt press of the powder, or in reflectance mode as a coating. For example, a neat press or a mixed lignin 16 with a potassium bromide or potassium chloride press may be used. With the use of infrared spectroscopy, total reflectance infrared spectroscopy, Raman spectroscopy, and/or total reflectance Raman spectroscopy for lignin 16 analysis, an analysis may be conducted in a relatively short period of time, generally about 15 minutes or less.

The molecular spectroscopy 18 may use one or more of these methods. If more than one of these methods may be used, then the methods may be completed in any order. One or more of the molecular spectroscopy 18 methods may also be repeated. Also, the sequence of analysis may be conducted in any order; that being either to first conduct of at least one method of molecular spectroscopy 18 followed by at least one method of physical-chemical analysis 20. Alternatively, at least one method of physical-chemical analysis 20 may be conducted first, and thereafter at least one method of molecular spectroscopy 18.

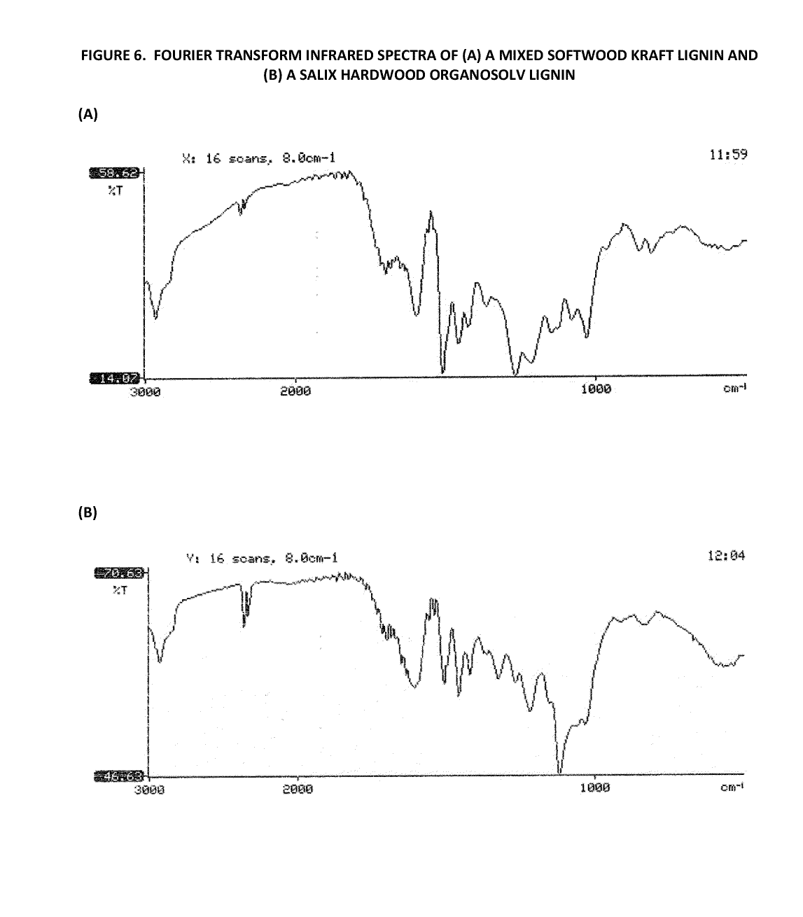

FIG. 6 provides an example of a Fourier transform infrared spectra of both a mixed softwood kraft lignin (shown in A) and an organosolv hardwood lignin from a Salix willow (shown in B). The use of a Fourier transform infrared spectra may be used to predict at least one biobased product from lignin 16. The infrared spectra of the corresponding lignin 16 samples were recorded using a Fourier transform infrared spectrometer. For the spectra provided in FIG. 6, a typical measurement was performed as a 16 scan data collection routine at 8 cm⁻¹ resolution and spectral window 4000-400 cm⁻¹. Other collection modes may also be applied to lignin spectroscopy.

Samples may be analyzed by this method as a solution, a powder, an oil mull, and/or as a pressed pellet. The actual samples of those shown in FIG. 6 were prepared as potassium bromide pellets which contained about 5% by weight lignin 16 to potassium bromide for reproducibility and/or consistency across spectra.

In performing a Fourier transform infrared spectra, an analysis sample may be prepared as a potassium bromide or potassium chloride press of lignin 16. An analysis sample preparation may also be as a neat press of lignin 16. Additionally, there may be no sample preparation wherein the infrared spectrum of a lignin 16 powder may be obtained by total reflectance infrared spectroscopy. Still another aspect of the process described herein is that the analysis may not require solubilisation of the lignin 16, which may allow a homogeneous analysis since lignin 16 may be sparingly soluble in many solvents. This analysis may use only a small amount of sample, such as a few milligrams.

With the Fourier transform infrared spectra, the analysis time of a sample may be on the order of 30 minutes or less. The analysis time of a sample may also be on the order of 15 minutes or less. In the process described herein, the infrared spectrum of lignin 16 may be recorded as a hardcopy spectrum or as an electronic file. The infrared spectrum of lignin 16 may also be analyzed manually or electronically, with the spectral data from the analysis of the infrared spectrum of lignin 16 input either manually or electronically into a secondary data analysis 22 method.

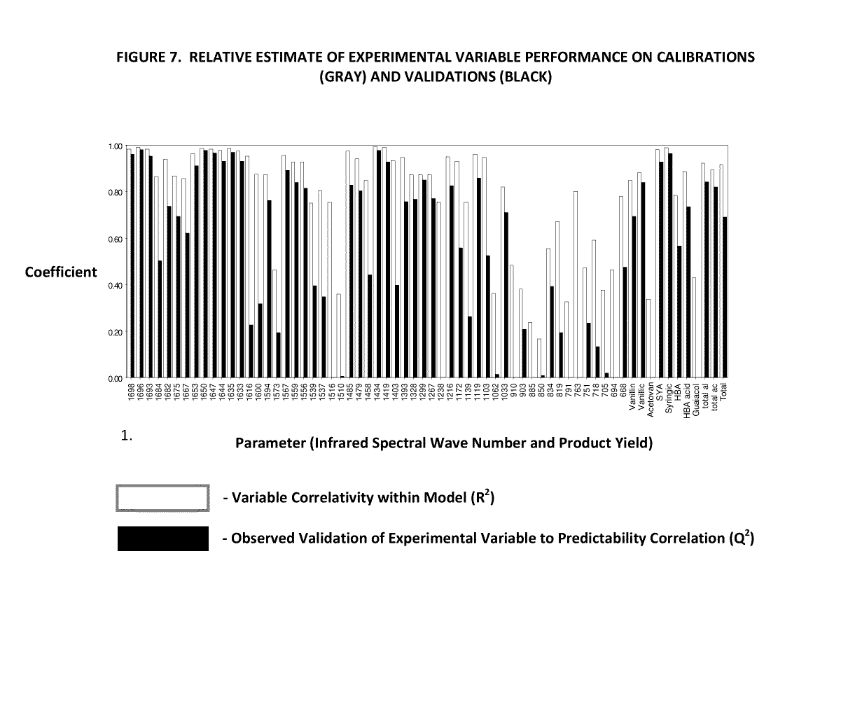

FIG. 7 provides an example showing a relative estimate of experimental variable performance on calibrations and validations within the method described herein. This experimental variable performance within the model is displayed as the gray bars and the observed validation of the experimental variable to predict correlation is displayed as black bars in FIG. 7. This correlation of a measured experimental variable (black bars provide infrared spectrum wave number and/or biobased product yields) relative to the correlation of that variable to an observed response (gray bars) may be high. These experimental variables provided in FIG. 7 where the gray and black bars may be of similar intensity may suggest the highest correlation in the validation between an observed response and a measured experimental variable.

For the process described herein, analyzing the infrared spectrum of lignin 16 may provide at least one experimental datum related to the molecular structure of the lignin 16. Also, analyzing the infrared spectrum of lignin 16 may provide at least two experimental data related to the molecular structure of the lignin 16. The analysis of lignin 16 may provide at least one experimental datum related to the p-coumaryl alcohol content, the coniferyl alcohol content, and the sinapyl alcohol content of the lignin 16. As well, analyzing lignin 16 may provide at least two experimental data related to the p-coumaryl alcohol content, the coniferyl alcohol content, and the sinapyl alcohol content of the lignin 16. Additionally, the experimental data from such analysis of lignin 16 may be processed manually or electronically.

In the analysis of lignin 16, the additional step of regression analysis may be used. The regression analysis of the experimental data may be provided by at least one regression analysis of linear regression analysis and/or multivariate regression analysis. In one embodiment of the process described herein, the regression analysis may be provided by linear least squares regression analysis.

In statistics, linear regression can be an approach to modeling the relationship between a scalar dependent variable, Y, and one or more explanatory variables, denoted as X. The case of one explanatory variable may be called simple regression, while using more than one explanatory variable may be multiple regressions. The linear regression described herein may be distinguished from multivariate linear regression, where multiple correlated dependent variables may be predicted, rather than a single scalar variable.

In linear regression, data may be modeled using linear predictor functions, and unknown model parameters may be estimated from the data in a process called linear models. Most commonly, linear regression may refer to a model in which the conditional mean of Y given the value of X is a function of X. Less commonly, linear regression may refer to a model in which the median, or some other quintile, of the conditional distribution of Y given X is expressed as a linear function of X Like all forms of regression analysis, linear regression may focus on the conditional probability distribution of Y given X, rather than on the joint probability distribution of Y and X, which is the domain of multivariate analysis. Since linear models may be easier to fit than models which are non-linearly related to their parameters, their statistical properties of the resulting estimators may be easier to determine.

Most applications of linear regression fall into one of the following two broad categories:
1. If the goal is prediction, or forecasting, linear regression may be used to fit a predictive model to an observed data set of Y and X values. After developing such a model, if an additional value of X is then given without its accompanying value of Y, the fitted model may be used to make a prediction of the value of Y.
2. Given a variable Y and a number of variables X₁, . . . , Xₚ that may be related to Y, linear regression analysis may be applied to quantify the strength of the relationship between Y and the Xⱼ, to assess which Xⱼ may have no relationship with Y at all, and to identify which subsets of the Xⱼ contain redundant information about Y.
   Linear regression models may often be fitted using the least squares approach, but they may also be fitted in other ways, such as by minimizing the “lack of fit” in some other norm (as with least absolute deviations regression), or by minimizing a penalized version of the least squares loss function as in ridge regression. Conversely, the least squares approach may be used to fit models that are not linear models. Thus, while the terms “least squares” and “linear model” are closely linked, they are not synonymous.

In the process described herein, the regression analysis may be provided by at least one computational method of manual computation, spreadsheet computation, website computation, and/or statistical software computation. The regression analysis may be conducted on a calculator or a computer.

Also, a partial least squares regression may be used in the process described herein. Partial least squares (PLS) regression is a statistical method that bears some relation to principal components regression; instead of finding hyperplanes of minimum variance between the response and independent variables, it finds a linear regression model by projecting the predicted variables and the observable variables to a new space. Because both the X and Y data are projected to new spaces, the PLS family of methods are known as bilinear factor models. Partial least squares Discriminant Analysis (PLS-DA) is used when Y is binary. PLS may be used to find the fundamental relations between two matrices (X and Y), i.e. a latent variable approach to modeling the covariance structures in these two spaces. A PLS model may also try to find the multidimensional direction in the X space that explains the maximum multidimensional variance direction in the Y space. PLS regression may be particularly suited when the matrix of predictors has more variables than observations, and when there is multi-collinearity among X values. By contrast, standard regression may fail in these cases.

With reference to FIG. 7, a partial least squares regression analysis model (PLS model) may be employed using raw infrared spectral data or preprocessed spectral data including, but not limited to smoothing, first and second derivative, baseline correction, standard normal variate, detrending, unit vector normalization, multiplicative signal correction, orthogonal signal correction as X matrix and/or chemical determination of the structure, including product distribution yields as the Y matrix. For this statistical process, X may be a measurable experimental variable, and Y may be the response variable associated with X. Within the process described, an X value from a new lignin 16 may correlate to a Y and may permit prediction of an outcome in advance of production operations.

In the example shown in FIG. 7, the data of X and Y matrices may be mean-centered and variance-scaled prior to PLS calculations if applicable. A cross-validation technique may be used to assess the model performance. From this regression analysis, a set of about 20 to about 40 experimental variables may emerge for use in reliably predicting lignin structural correlations, lignin composition, biobased products identities, and/or biobased product yields. Infrared spectrum of a new lignin may then be analyzed by this model to predict suitability for use in biobased product production (i.e., quality control), to determine similarity to other lignins, and/or to predict biobased product identities and/or product yields in advance of production operations.

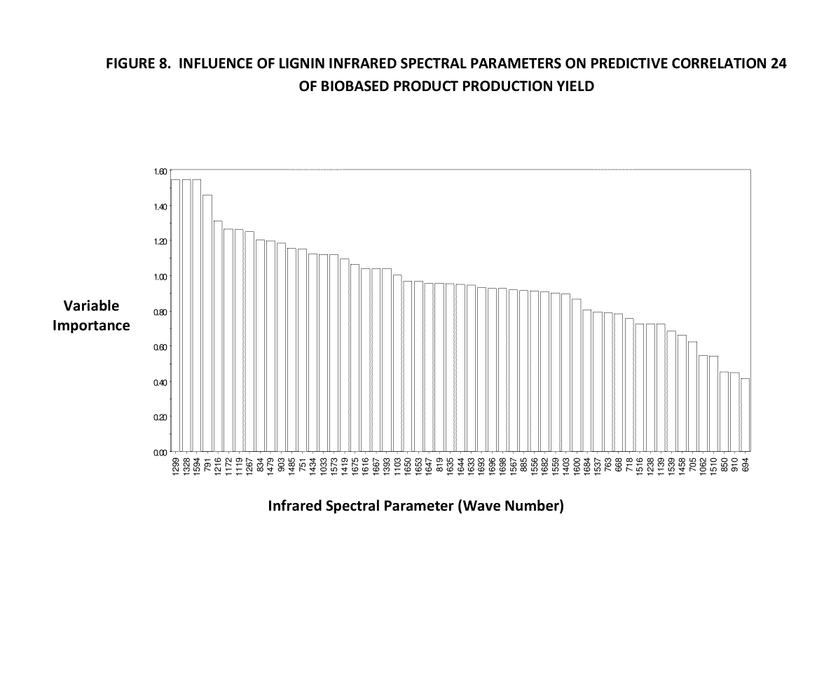

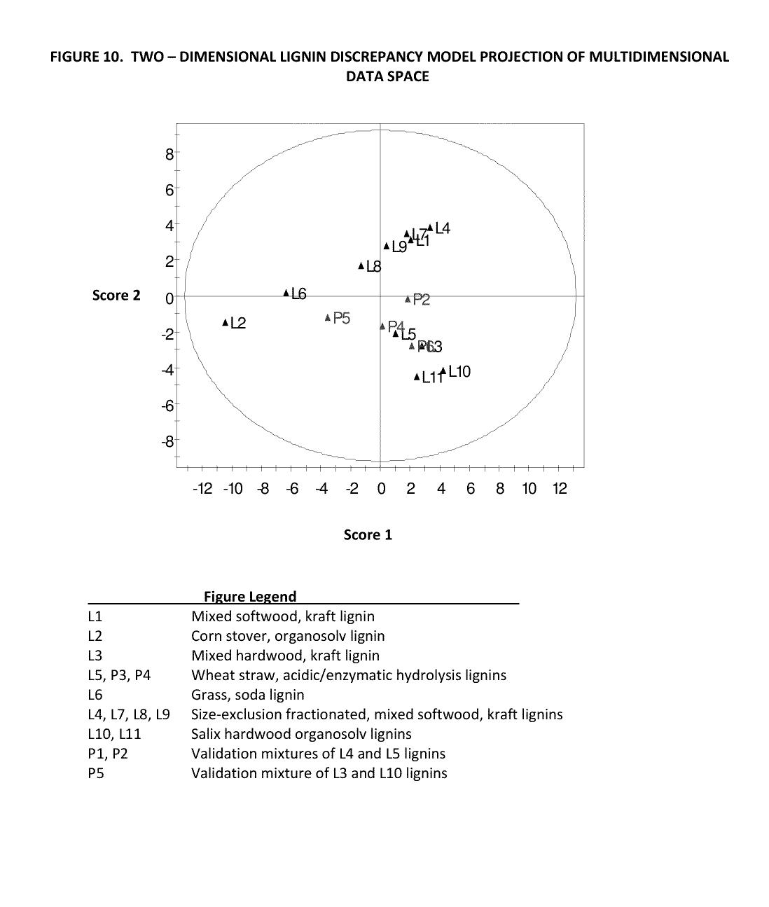

FIG. 8 shows the influence of lignin infrared spectral parameters on the predictive correlation 24 of biobased product production yield in the determination of at least one biobased product. In this particular instance in FIG. 10, about 55 experimental variables X where X may be lignin 16 infrared spectral wavelengths in the region of about 1800 cm⁻¹ to about 600 cm⁻¹ may be plotted against a response variable Y where Y may be the product yield from biobased product production.

With the graphical method shown in FIG. 8, assessing the influence of a specific experimental variable X upon a specific response variable Y may be applied to an X derived from at least one analysis method of molecular spectroscopy 18 and/or physical-chemical analysis 20 shown in FIGS. 5 and 9. For each analysis method, a unique set of experimental variables X may be identified as correlating best with a specific response variable Y, and the influence of an experimental variable X upon the response variable Y may be specific to that model.

The experimental variable importance projected (VIP) model space shown in FIG. 10 may be the sum over all model dimensions for the contributions of that experimental variable's influence upon the response variable. FIG. 8 may also provide for a comparison of the VIP of one experimental variable to the VIP of the other experimental variables in a model. In this comparison, those experimental variables X having a VIP of about 1 or greater, may be relevant for predicting a response variable Y. Within FIG. 8, approximately 22 of the 50 original experimental variables shown in this example can have a VIP of about 1 or greater. That subset of 22 of 50 experimental variables can thus form the basis for a model that may permit the practitioner to predict product yield of at least one biobased product from a new lignin 16 prior to production operations. Furthermore, the model may permit the practitioner to predict product yields of at least two biobased products from a new lignin 16 prior to production operations. That prediction may be based solely on specific experimental variables X (i.e., specific infrared spectrum wavelengths) that have been demonstrated to have the greatest influence on the response variable Y (i.e., the product yield). In the ability to predict the product yield of at least one biobased product produced from the lignin 16, the step of determining the composition of the lignin provides the prediction of a product yield of at least one biobased product produced from the lignin with an accuracy of about 70% to about 100%. Additionally, the step of determining the composition of the lignin provides the prediction of a product yield of at least one biobased product produced from the lignin with an accuracy of about 85% to about 100%.

FIG. 9 may provide methods for the physical-chemical analysis 20 for lignin 16 analysis in the determination of at least one biobased product from at least one lignin 16 source. The physical-chemical analysis 20 may use one or more of these methods. If more than one of these methods may be used, then the methods may be completed in any order. One or more of these physical-chemical analysis 20 methods may be repeated. This physical-chemical analysis 20 may also comprise at least one method of physical analysis methods 34 and/or chemical analysis methods 36. Furthermore, the physical-chemical analysis 20 may comprise at least two methods of physical analysis methods 34 and/or chemical analysis methods 36. Additionally, at least one physical-chemical analysis 20 method may be conducted along with at least one method of molecular spectroscopy 18. Also, the sequence of analysis may be conducted in any order; that being either to first conduct at least one method of molecular spectroscopy 18 followed by at least one method of physical-chemical analysis 20. Alternatively, at least one method of physical-chemical analysis 20 may be conducted first, and thereafter at least one method of molecular spectroscopy 18. Also, one or more of the physical-chemical analysis 20 methods may be repeated.

The physical analysis methods 34 provided in FIG. 9 are methods of physical-chemical analysis 20 that may determine at least one physical property or characteristic of lignin 16. These analytical methods and associated lignin property/characteristic may include those listed in Table B:

TABLE B
Lignin Property/Characteristic For Physical Analysis Methods 34

| PHYSICAL ANALYSIS | LIGNIN 16 PHYSICAL |
|---|---|
| METHODS 34 | PROPERTY/CHARACTERISTIC |
| Appearance analysis | Color and form |
| Moisture content analysis | Moisture content per unit weight |
| Melting point analysis | Melting point or decomposition |
| | temperature |
| Melting point range analysis | Melting point or decomposition |
| | temperature range |
| Molecular weight analysis | Molecular weight |
| Molecular weight distribution | Molecular weight distribution |
| analysis | |
| Size exclusion | Molecular weight range |
| chromatographic analysis | |
| Particle size analysis | Particle size dimension |
| Thermogravimetric analysis | Degradation temperature and purity |
| Differential scanning | Fusion and crystallization events; |
| calorimetric analysis | glass transition temperature |
| Dynamic mechanical analysis | Glass transition temperature |
| Rheological analysis | Viscoelastic properties |
| Density analysis | Solid density and/or volume |
| | displacement per unit weight |
| X-ray diffraction powder | Phase identification, phase |
| analysis | transitions, bulk modulus, expansion |
| | tensors |
| Heavy metals analysis | Heavy metal content per unit weight |
| Elemental analysis | Carbon, hydrogen and oxygen |
| | abundance per unit weight |
| pH titration analysis | Equivalents of free phenolic and |
| | carboxylic acid groups per unit weight |
| Residue on ignition analysis | Non-combustible content, primarily |
| | inorganics, per unit weight |
| Thin layer liquid | Separation and identification of soluble |
| chromatographic analysis | lignin fragments and products/degradants |
| High performance liquid | Separation and identification of soluble |
| chromatographic analysis | lignin fragments and products/degradants |
| Gas chromatographic analysis | Separation and identification of volative |
| | lignin fragments and products/degradants |

Chemical analysis methods 36 are methods of physical-chemical analysis 20 that can be based on determining at least one chemical property/characteristic of lignin 16. The chemical analysis methods 36 may comprise at least one treatment method of chemical treatment, biological treatment, photochemical treatment, and/or thermal treatment.

For chemical treatment methods of chemical analysis methods 36, at least one method of chemical oxidation analysis, chemical derivative analysis, chemical methoxy content analysis, chemical degradation analysis, and/or residual carbohydrate analysis may be included. In one type of chemical analysis method 36, chemical oxidation analysis is a method of chemical analysis methods 36 that may facilitate the measurement of the p-coumaryl alcohol, coniferyl alcohol, and sinapyl alcohol content of the lignin 16. The method may generally be based on an exhaustive oxidation of lignin 16 to a mixture of aryl carboxylic acids and/or aryl aldehydes. Common oxidants for this method may comprise but are not limited to permanganate and nitrobenzene. The aryl carboxylic acids and/or aldehydes may be analyzed by physical-chemical analysis 20 and/or molecular spectroscopy 18 methods described herein to determine identity and relative ratios/quantity. Chemical derivative analysis is a method of chemical analysis methods 36 wherein lignin 16 may be chemically altered in some manner. In another type of chemical analysis method 36, chemical derivative analysis may comprise, but is not limited to, at least one method of esterification, O-methylation, and/or O-acetylation to produce a derivative product of lignin 16. Derivatives of lignin 16 may be analyzed by physical-chemical analysis 20 and/or molecular spectroscopy 18 methods described herein to determine the phenolic content or extent of free hydroxyl groups. In yet another type of chemical analysis method 36, chemical methoxy content analysis is a method of chemical analysis methods 36 wherein lignin 16 may be oxidized, for example with hydroiodic acid. Methoxy content may correlate with the yield of methyl iodide formed in the hydroiodic acid reaction. The methyl iodide may generally be measured by gas chromatographic analysis methods. In still another type of chemical treatment method, chemical degradation analysis is a method of chemical analysis methods 36 wherein lignin 16 may be degraded by chemical means to produce degradant products of lignin 16. Chemical oxidation analysis may be considered as one method of chemical degradation analysis. Another method of chemical degradation analysis may be phenolic hydroxyl content by periodate oxidation in acetic acid. Still other chemical means to degrade lignin may comprise, but are not limited to, at least one method of caustic treatment, acid treatment, free radical processes, and/or reducing chemical environments. The degradant products of chemical degradation analysis may be analyzed by physical-chemical analysis 20 and/or molecular spectroscopy 18 methods described herein to determine identity and amount. In still yet another type of chemical treatment method, residual carbohydrate analysis is a method of chemical analysis methods 36 wherein residual cellulosic or hemicellulosic sugars bound to lignin 16 may be measured. Often the procedure may be based on acidic hydrolysis and high performance liquid chromatography analysis.

Besides those methods for chemical analysis methods 36 described above, biodegradation analysis, photochemical degradation analysis and pyrolysis degradation analysis are other methods of chemical analysis methods 86 wherein lignin 16 may be degraded by enzymes (either isolated or whole cell), light (photolysis) or heat, respectively. The degradant products of these methods may then be analyzed by physical-chemical analysis 20 and/or molecular spectroscopy 18 methods described herein to determine identity and amount.

For the process described herein, the physical-chemical analysis 20 may comprise at least one physical analysis methods 34 of appearance analysis, moisture content analysis, density analysis, and/or residue on ignition analysis. The physical-chemical analysis 20 may also comprise at least one chemical analysis methods 36 of chemical oxidation analysis, chemical derivative analysis, chemical methoxy content analysis, and/or residual carbohydrate analysis.

FIG. 10 provides a two-dimensional projection on the experimental variable importance projected (VIP) model space for the method described wherein the composition of the lignin 16 may be graphically represented. FIG. 10 may display a cross cut of the three-dimensional component scores of each lignin 16. Different two-dimensional cross cuts of the three-dimensional space may show similar results. Structural discrimination between the different lignins may be represented by a projection of the component scores of each lignin on three-dimensional model space. FIG. 10 demonstrates that the model can effectively discriminate between structural differences across the different lignins according to the experimental spectral data. This may be shown by different lignins occupying positions in different three-dimensional space of the model.

Within the three-dimensional projection, those lignins of greatest similarity may lie closest to each other in the model space projection of FIG. 10. For example, lignins L1 and L2 are widely different materials (i.e., derived from corn stover versus mixed softwood, and prepared by organosolv versus kraft processes, respectively). These differences may be reflected by their respective component scores occupying different quadrant spaces of the model. This projection may be consistent with the known significant differences in H:G:S content between a corn stover lignin and a softwood lignin. Within the example, Lignins L10 and L11 are organosolv hardwood lignins derived from a Salix willow species by an OrganoSolv™ technology. The component scores for these lignins may lie in close proximity to each other and within the same quadrant space of the figure. In FIG. 10, Lignins L1, L4, L7, L8 and L9 are mixed softwood kraft lignins. One difference between these lignins may be the degree to which the lignin may have been fractionated according to molecular weight distribution. Because each of these lignins may originate from a softwood origin of high G content, their respective component scores may fall near each other, although these scores may not be identical to each other because of physical differences in each sample. In another instance, Lignins L3 and L10/L11 are hardwood lignins in which the former lignin is from a kraft mill process and the latter two lignins are from OrganoSolv™ processes. Because hardwood lignins may have a higher S content than softwoods, the component scores of these lignins may be well separated in three-dimensional space from the softwood lignins. Moreover, because the S:G contents of hardwoods may be similar, these lignins may lie in the same quadrant space of the VIP model space.

From this model, the H:G:S structural composition of an unknown lignin may be predicted from the model space position the unknown lignin occupies relative to that of the known lignins. These structural composition analyses may in turn allow a practitioner to reliably predict the biobased product composition from a specific lignin 16 or lignin blend in advance of a production operation. Moreover, analyzing lignin 16 by this model may provide for prediction of the product identity of at least one biobased product that may be produced from the lignin 16. Furthermore, analyzing lignin 16 by this model may provide for prediction of the product identity of at least two biobased products that may be produced from the lignin 16. Using this model, the step of using the determination of the composition to predict at least one biobased product produced from the lignin may have an accuracy of about 70% to about 100%. Additionally, step of using the determination of the composition to predict at least one biobased product produced from the lignin may have an accuracy of about 85% to about 100%. In addition, the structural analysis may be used in raw material quality control operations in a biorefinery so as to assure reliable biobased product production. In analogous manner, a model may be developed to predict product yield of at least one biobased product from lignin 16.

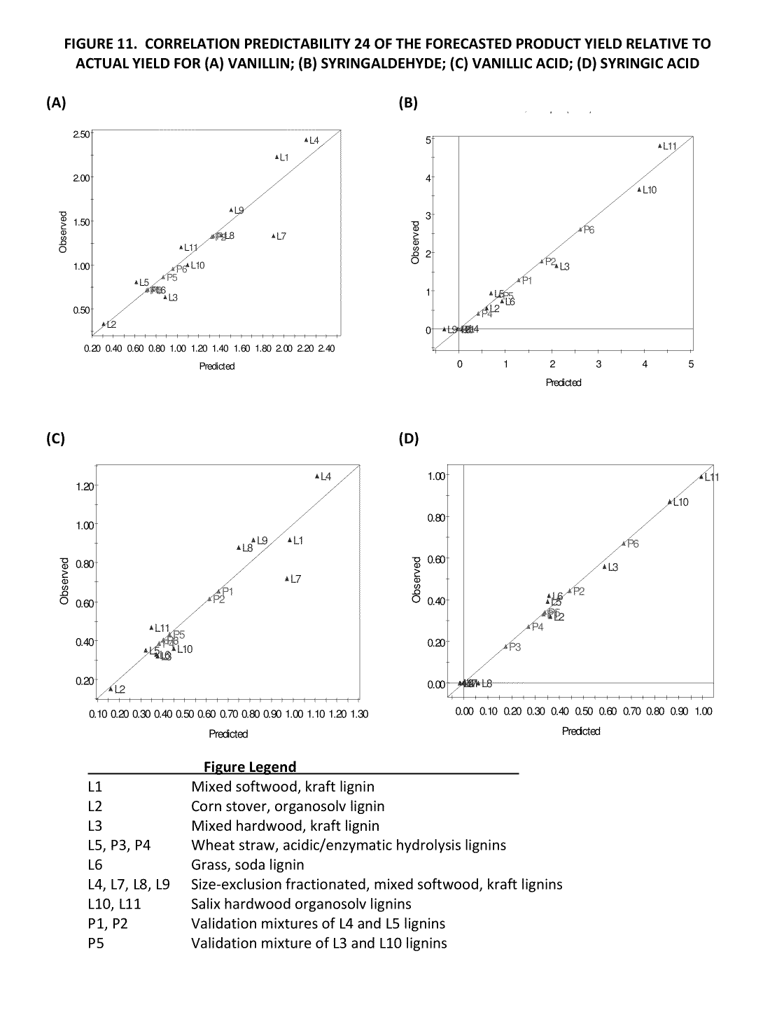

FIG. 11 provides examples for the correlation predictability 24 of the forecasted product yield relative to the actual yield in the determination of at least one biobased product from lignin 16 for the method described herein. Besides the prediction of at least one biobased product from lignin 16, the method may also provide a prediction of the yield from the production of at least one biobased product. These plots provided in FIG. 11 may show the observed response values Y versus the fitted or predicted response values Y from the model for product yields of (A) vanillin, (B) syringaldehyde, (C) vanillic acid, and (D) syringic acid.

Plots with points close to a diagonal line Y_{pred}=Y_{obs} may support a model that is a good predictor of experimental outcome. The data points in FIG. 11 that begin with the letter L are the values for the standard samples of lignin 16 used in the model development, whereas the points that begin with the letter P are for the unknown samples. The data within FIG. 11 may illustrate a strong support of the model to accurately predict product yield on the basis of lignin 16 infrared spectral wavelength experimental response X relative to response variable Y. Additional lignin 16 samples used in this correlation predictability 24 may assist to expand and better qualify the influence each X, has on product yield. The correlation predictability 24 may also be extended to assessing the influence of a specific experimental variable X upon a specific response variable Y which may be applied to an X derived from at least one analysis method of molecular spectroscopy 18 and/or physical-chemical analysis 20, as shown in FIGS. 5 and 9. For each analysis method, a unique set of experimental variables X may be identified as correlating best with a specific response variable Y. In other words, the influence of an experimental variable X upon the response variable Y may be specific to that model.

These plots for the correlation predictability 24 of the product yield relative to the actual yield may demonstrate the ability to predict the product yield of at least one biobased product produced from lignin 16 or a blended lignin 16 by such a model. In the ability to predict the product yield of at least one biobased product produced from the lignin 16, the step of determining the composition of the lignin provides the prediction of a product yield of at least one biobased product produced from the lignin with an accuracy of about 70% to about 100%. Additionally, the step of determining the composition of the lignin provides the prediction of a product yield of at least one biobased product produced from the lignin with an accuracy of about 85% to about 100%. With this model for the process described herein, both the identity and yield of at least one biobased product may be predicted prior to the actual production run.

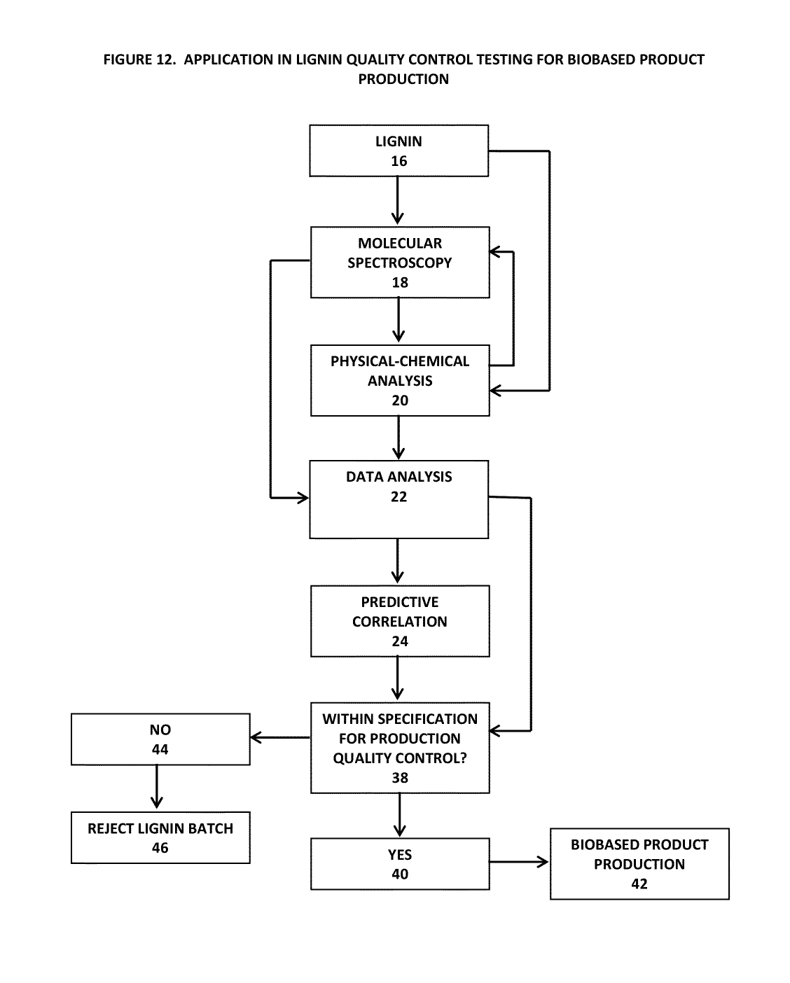

FIG. 12 provides an application of lignin quality control testing as a raw material for biobased product production 42. As previously described in FIG. 1, a predictive correlation 24 may be provided from the method described, wherein data analysis 22 of experimental data obtained by analyzing the lignin 16 can provide the ability to predict the product identity and/or product yield of at least one biobased product that may be produced from the lignin 16.

Within the process described herein, a predictive correlation 24 may be provided from the data analysis 22 of experimental data obtained from at least one method of molecular spectroscopy 18 and/or at least one method of physical-chemical analysis 20. By analyzing the lignin 16, the ability to predict the product identity of at least one biobased product that may be produced from the lignin 16 may be provided. Furthermore, the predictive correlation 24 may provide the ability to predict the product identities of at least two biobased products that may be produced from the lignin 16. Moreover, the step of using the determination of the composition to predict at least one biobased product produced from the lignin may have an accuracy of about 70% to about 100%. Additionally, the step of using the determination of the composition to predict at least one biobased product produced from the lignin may have an accuracy of about 85% to about 100%. Within the process described herein, a predictive correlation 24 step may be provided from the lignin 16 analysis method, wherein data analysis 22 of experimental data obtained by analyzing the lignin 16 can provide the ability to predict the product yield of at least one biobased product produced from the lignin 16. In another embodiment, the predictive correlation 24 step may provide the ability to predict the product yields of at least two biobased products that may be produced from the lignin 16. Furthermore, in the ability to predict the product yield of at least one biobased product produced from the lignin 16, the step of determining the composition of the lignin provides the prediction of a product yield of at least one biobased product produced from the lignin with an accuracy of about 70% to about 100%. Additionally, the step of determining the composition of the lignin provides the prediction of a product yield of at least one biobased product produced from the lignin with an accuracy of about 85% to about 100%.

The use of the lignin evaluation method described herein may offer the benefit of providing quality control information related to (1) lignin structural correlation with other lignin 16, (2) the p-coumaryl alcohol content, the coniferyl alcohol content, and the sinapyl alcohol content of lignin 16, (3) the identity of at least one biobased product that may be produced from lignin 16, and/or (4) the product yield of at least one biobased product that may be produced from lignin 16. Collectively, this information may be used to monitor quality control of lignin in at least one operation of a biobased product refinery operation, commercial biomass fractionators 06 operation, pulp/paper mills 08 operation, cellulosic ethanol refineries 10 operation, and/or sugar cane mill 12 operations. Such quality control may be achieved by determining whether data from the data analysis 22 step and/or the predictive correlation 24 step are within specification for production quality control 38. If the data analysis 22 step and/or the predictive correlation 24 step are within specification for production quality control 38 (referred to as Yes 40), then quality control may move the lignin 16 to biobased product production 42. If the data analysis 22 step and/or the predictive correlation 24 step are not within specification for production quality control 38 (referred to as No 44), then quality control may reject lignin batch 46 and not advance the lignin 16 to biobased product production 42. Overall, a method of analysis of lignin 16 may require less than about 2 hours to perform. It may also require less than about 30 minutes to perform. Furthermore, a method of analysis of lignin 16 may be automated.

Within the process described herein, the biobased product production 42 may be provided by at least one process of chemical processing, catalytic processing, biological processing, and/or pyrolytic processing. The biobased product production 42 may also provide at least one product of biobased chemicals, biofuels, biobased materials, and/or lignin residues. These biobased chemicals may comprise at least one chemical of commodity chemicals, fine chemicals, specialty chemicals, and/or performance chemicals. The biobased chemicals may also comprise at least one chemical of achiral chemicals, racemic chemicals, and/or chiral chemicals. Specifically, the biobased chemicals may comprise at least one chemical of aryl aldehydes, aryl carboxylic acids, aryl esters, aryl ketones, aryl alcohols, aliphatic carboxylic acids, phenols, alkyl phenols, alkenyl phenols, benzene, toluene, xylenes, mesitylenes, biaryls, aryl alkanes, aryl alkenes, alkanes, alkenes, cycloalkanes, cycloalkenes, alkyl esters, performance chemicals, and/or pyrolysis oils. Likewise, performance chemicals may comprise at least one chemical of phenols, alkyl phenols, alkenyl phenols, benzene, toluene, xylenes, mesitylenes, biaryls, aryl alkanes, aryl alkenes, alkanes, alkenes, cycloalkanes, cycloalkenes, and/or alkyl esters. Further, biofuels may comprise at least one biobased chemical of alkanes, alkenes, cycloalkanes, cycloalkenes, alkyl esters, benzene, toluene, xylenes, mesitylenes, biaryls, aryl alkanes, aryl alkenes, alkyl naphthalenes, phenols, alkyl phenols, alkenyl phenols, and/or pyrolysis oils.

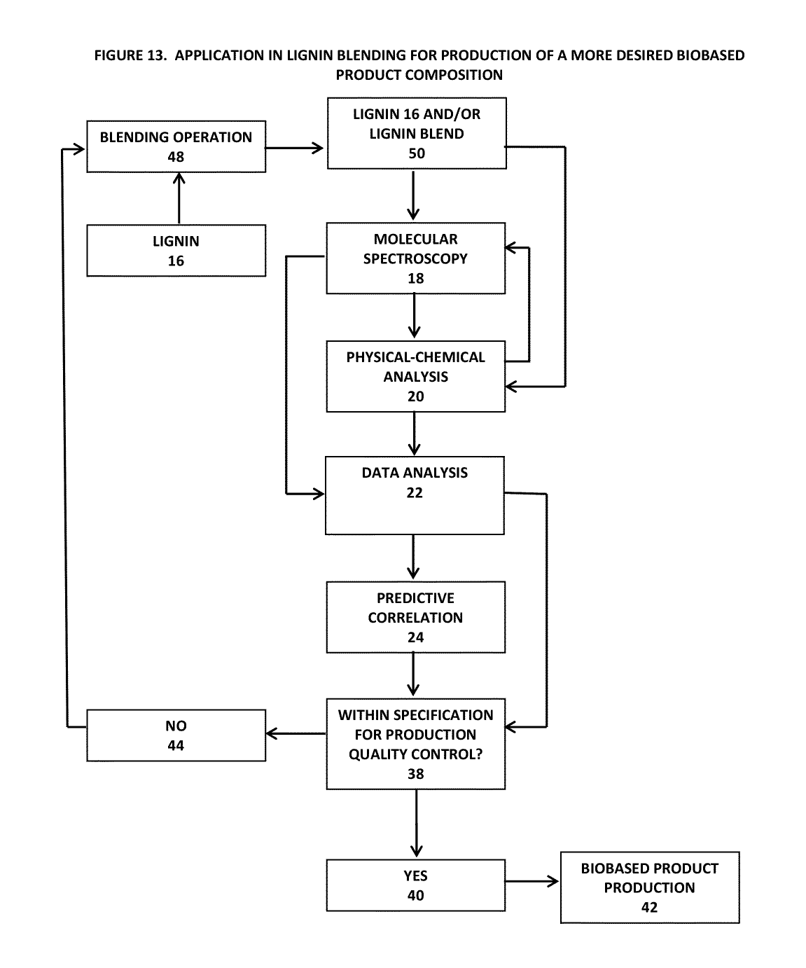

FIG. 13 provides an application of lignin blending as a raw material for production of a more desired biobased product composition. The process described in FIG. 13 may be used where 1) lignin 16 is rejected from the process, as described in FIG. 12, and/or 2) a particular biobased product is desired. Where the lignin 16 still does not meet the specifications for quality control, the lignin 16 may still be processed so that at least one biobased product may be produced from the lignin 16. For the production of a particular biobased product, such blending may offer the advantage of producing a lignin 16 of preferred characteristics for biobased product production.

Within this method, at least two lignin 16 may be received. The lignin may be lignin 16 new to the process, which can be referred together as the lignin blend 50. If one of the lignin 16 within the process was rejected as to being within specification for production quality control? 38 (referred to as No 44 within the figure), then it may be blended with another lignin 16 or lignin blend 50 in order to provide at least one biobased product. These blends may be designed to produce a specific biobased product and/or to eliminate waste from the process described herein.

The quality of each lignin 16 may be first evaluated and analyzed, as described in FIG. 12. When the lignin 16 reaches the step on whether it is within specification for production quality control 38, it may undergo lignin 16 blending if the lignin 16 evaluated is not within specification (referred to as No 44 in FIG. 12). The blending shown in FIG. 13 provides an alternative to reject lignin batch 46 shown in FIG. 12.

At least two different lignin 16 types may then transfer to a blending operation 48. The lignin 16 within the process shown in FIG. 13 may be lignin 16 that had been rejected (i.e. a No 44 response) as to whether the lignin 16 was within specification for production quality control? 38, and/or it may be fresh lignin 16 added to the process. No matter the origin, when at least two lignin 16 sources are added to the process, a lignin blend 50 can result. Within the process, lignin 16 in the blending operation 48 may be a solid or a solution from which a lignin blend 50 may be obtained. If the lignin blend 50 may be in solution, a solid lignin blend 50 may be obtained by methods described in utility applications commonly owned by the current assignee of the instant application, and are incorporated herein. The blending operation may mechanically mix at least two different lignin 16 types within the method described herein.

Data analysis 22 of the lignin blend 50 may further include regression analysis, wherein the regression analysis of the experimental data may be provided by at least one regression analysis method of linear regression analysis and/or multivariate regression analysis. Regression analysis of the experimental data may be comprised of at least one computational method of manual computation, spreadsheet computation, website computation, and/or statistical software computation. Also, regression analysis of the experimental data may be conducted on a calculator or a computer. In one embodiment of the method, a predictive correlation 24 may be provided from the lignin blend 50 analysis method, wherein data analysis 22 of experimental data obtained by analyzing the lignin blend 50 may provide the ability to predict the product identity of at least one biobased product that may be produced from the lignin blend 50. In another embodiment of the method, the predictive correlation 24 may provide the ability to predict the product identities of at least two biobased products that may be produced from the lignin blend 50. In yet another embodiment, the step of using the determination of the composition to predict at least one biobased product produced from the lignin may have an accuracy of about 70% to about 100%. In still another embodiment, the step of using the determination of the composition to predict at least one biobased product produced from the lignin may have an accuracy of about 85% to about 100%.

Data analysis 22 of the lignin blend 50 may further include a predictive correlation 24 step that may be provided from the lignin blend 50 analysis method, wherein data analysis 22 of experimental data obtained by analyzing the lignin blend 50 may provide the ability to predict the product yield of at least one biobased product produced from the lignin blend 50. In another embodiment, the predictive correlation 24 step may provide the ability to predict the product yields of at least two biobased products that may be produced from the lignin blend 50. The step of determining the composition of the lignin may provide the prediction of a product yield of at least one biobased product produced from the lignin with an accuracy of about 70% to about 100%. Additionally, the step of determining the composition of the lignin may provide the prediction of a product yield of at least one biobased product produced from the lignin with an accuracy of about 85% to about 100%.

The use of the lignin evaluation method may offer the benefit of providing quality control information related to: (1) lignin structural correlation of the lignin blend 50 relative to other lignin 16, (2) the p-coumaryl alcohol, coniferyl alcohol, and sinapyl alcohol content of lignin blend 50 as well as the linkages for the lignin 16, (3) the identity of at least one biobased product that may be produced from lignin blend 50, and/or (4) the product yield of at least one biobased product that may be produced from lignin blend 50. Collectively, this information may be used to monitor the quality of lignin in at least one operation of a biobased product refinery operation, commercial biomass fractionators 06 operation, pulp/paper mills 08 operation, cellulosic ethanol refineries 10 operation, and/or sugar cane mill 12 operations. Such quality control may be achieved by determining whether data from the data analysis 22 step and or the predictive correlation 24 step are within specification for production quality control? 38. If the lignin 16 or lignin blend 50 is within the specification desired (referred to as Yes 40), then the lignin 16 may be processed to biobased product production 42. If not (referred to as No 44), then the lignin blend 50 may be reprocessed further in the blending operation 48. Moreover, such blending of lignin 16 to produce a lignin blend 50 that is within specification for production quality control 38 may offer additional benefits which may include: (1) potentially accepting a lignin 16 into production operations that otherwise would not provide the desired biobased product composition and would constitute a reject lignin batch 46, and/or (2) controlling the biobased product composition and/or biobased product yield that may be achieved in a biobased product refinery operation.

Further, a method of analysis of lignin blend 50 may require less than about 2 hours to perform. A method of analysis of lignin blend 50 may also require less than about 30 minutes to perform. A method of analysis of lignin blend 50 can be automated.

Further, one or more of the biobased products from the biobased product production 42 may be provided by at least one process of chemical processing, catalytic processing, biological processing, and/or pyrolytic processing. One or more biobased products may be comprised of at least one product of biobased chemicals, biofuels, biobased materials, and/or lignin residues. Moreover, one or more biobased chemicals may comprise at least one chemical of commodity chemicals, fine chemicals, specialty chemicals, and/or performance chemicals. The biobased chemicals may also comprise at least one chemical of achiral chemicals, racemic chemicals, and/or chiral chemicals. The production of one or more biobased products is detailed further by applications commonly owned by the assignee of the instant application, which are incorporated within the present application.

The embodiments have been described, hereinabove. It will be apparent to those skilled in the art that the above methods and apparatuses may incorporate changes and modifications without departing from the general scope of this method. It is intended to include all such modifications and alterations in so far as they come within the scope of the appended claims or the equivalents thereof.

Having thus described the method, it is now claimed:

1. A method for evaluation of lignin, comprising the steps of:
providing lignin from a source, wherein the composition of said lignin is unknown;
analyzing said lignin;
determining said composition of said lignin; and
using said determination of said composition to predict at least one biobased product produced from said lignin.

2. The method of claim 1, wherein said lignin is provided from at least one biomass of plant biomass, woody plant biomass, agricultural plant biomass, and cultivated plant biomass.

3. The method of claim 1, wherein said lignin is provided from at least one biomass of fresh plant biomass, recovered plant biomass, pulp and paper mill biomass, cellulosic ethanol refinery biomass, sugar cane mill biomass, kraft pulp mill, sulfite pulp mill, soda pulp mill, cellulosic ethanol refinery, commercial plant biomass fractionator biomass, and lignin residues biomass.

4. The method of claim 1, wherein said lignin is provided from waste lignin.

5. The method of claim 4, wherein said waste lignin is provided from at least one waste lignin of recovered biomass waste lignin, kraft pulp mill waste lignin, sulfite pulp mill waste lignin, soda pulp mill waste lignin, cellulosic ethanol refinery waste lignin, commercial plant biomass fractionator waste lignin, and sugar cane mill waste lignin.

6. The method of claim 1, wherein said lignin comprises at least one lignin building block of p-coumaryl alcohol, coniferyl alcohol, and sinapyl alcohol.

7. The method of claim 1, wherein said analyzing said lignin is provided by molecular spectroscopy.

8. The method of claim 7, wherein said molecular spectroscopy is provided by at least one molecular spectroscopy of infrared spectroscopy, total reflectance infrared spectroscopy, Raman spectroscopy, ultraviolet-visible spectroscopy, luminescence spectroscopy, fluorescence spectroscopy, mass spectroscopy, diffuse reflectance spectroscopy, transient spectroscopy, homonuclear magnetic resonance spectroscopy, heteronuclear magnetic resonance spectroscopy, and/or two-dimensional nuclear magnetic resonance spectroscopy.

9. The method of claim 7, wherein said molecular spectroscopy is provided by at least two molecular spectroscopies infrared spectroscopy, total reflectance infrared spectroscopy, Raman spectroscopy, ultraviolet-visible spectroscopy, luminescence spectroscopy, fluorescence spectroscopy, mass spectroscopy, diffuse reflectance spectroscopy, transient spectroscopy, homonuclear magnetic resonance spectroscopy, heteronuclear magnetic resonance spectroscopy, and/or two-dimensional nuclear magnetic resonance spectroscopy.

10. The method of claim 1, wherein said analyzing said lignin is provided by a physical-chemical analysis.

11. The method of claim 10, wherein said physical-chemical analysis is provided by at least one physical-chemical analysis of appearance analysis, moisture content analysis, melting point analysis, melting point range analysis, molecular weight analysis, molecular weight distribution analysis, size exclusion chromatographic analysis, thin layer liquid chromatographic analysis, high performance liquid chromatographic analysis, gas chromatographic analysis, particle size analysis, chemical oxidation analysis, chemical derivative analysis, chemical methoxy content analysis, chemical degradation analysis, biodegradation analysis, photochemical degradation analysis, pH titration analysis, thermogravimetric analysis, differential scanning calorimetric analysis, dynamic mechanical analysis, rheological analysis, pyrolysis degradation analysis, residual carbohydrate analysis, residue on ignition analysis, heavy metals analysis, density analysis, x-ray diffraction analysis, x-ray powder analysis, and elemental analysis.

12. The method of claim 10, wherein said physical-chemical analysis is provided by at least two physical-chemical analyses of appearance analysis, moisture content analysis, melting point analysis, melting point range analysis, molecular weight analysis, molecular weight distribution analysis, size exclusion chromatographic analysis, thin layer liquid chromatographic analysis, high performance liquid chromatographic analysis, gas chromatographic analysis, particle size analysis, chemical oxidation analysis, chemical derivative analysis, chemical methoxy content analysis, chemical degradation analysis, biodegradation analysis, photochemical degradation analysis, pH titration analysis, thermogravimetric analysis, differential scanning calorimetric analysis, dynamic mechanical analysis, rheological analysis, pyrolysis degradation analysis, residual carbohydrate analysis, residue on ignition analysis, heavy metals analysis, density analysis, x-ray diffraction analysis, x-ray powder analysis, and elemental analysis.

13. The method of claim 1, wherein said analyzing said lignin provides at least one experimental datum related to said composition of said lignin.

14. The method of claim 13, wherein said composition of said lignin biomass comprises p-coumaryl alcohol, coniferyl alcohol, and sinapyl alcohol.

15. The method of claim 13, wherein said experimental datum is processed manually or electronically.

16. The method of claim 1, wherein said analyzing said lignin provides at least two experimental data related to the composition of said lignin.

17. The method of claim 1, wherein said analyzing said lignin comprises the additional step of:
performing a statistical analysis of experimental data.

18. The method of claim 17, wherein said step of performing statistical analysis of experimental data is provided by at least one computational method of manual computation, spreadsheet computation, website computation, and statistical software computation.

19. The method of claim 17, wherein said step of performing statistical analysis of experimental data is conducted on a calculator or a computer.

20. The method of claim 17, wherein said step of performing statistical analysis of experimental data comprises a regression analysis of experimental data.

21. The method of claim 20, wherein said regression analysis of experimental data is provided by at least one regression analysis of linear regression analysis and multivariant regression analysis.

22. The method of claim 20, wherein said regression analysis of experimental data is provided by linear least squares regression analysis.

23. The method of claim 20, wherein said regression analysis of experimental data is provided by at least one computational method of manual computation, spreadsheet computation, website computation, and statistical software computation.

24. The method of claim 20, wherein said regression analysis of experimental data is conducted on a calculator or a computer.

25. The method of claim 1, wherein said step of using said determination of said composition to predict at least one biobased product produced from said lignin has an accuracy of about 70% to about 100%.

26. The method of claim 1, wherein said step of using said determination of said composition to predict at least one biobased product produced from said lignin has an accuracy of about 85% to about 100%.

27. The method of claim 1, wherein said determination of said composition provides a prediction of a product yield of at least one of said biobased product produced from said lignin.

28. The method of claim 1, wherein said determination of said composition provides a prediction of a product yield of at least two of said biobased products produced from said lignin.

29. The method of claim 27, wherein said step of determining said composition of said lignin provides said prediction of a product yield of at least one of said biobased product produced from said lignin with an accuracy of about 70% to about 100%.

30. The method of claim 27, wherein said step of determining said composition of said lignin provides said prediction of a product yield of at least one of said biobased product produced from said lignin with an accuracy of about 85% to about 100%.

31. The method of claim 1, wherein said at least one biobased product produced from said lignin is provided by at least one product of biobased chemicals, biofuels, biomaterials, and lignin residues.

32. The method of claim 1, wherein said determining said composition of said lignin provides quality control in at least one operation of a biobased product refinery operation, commercial biomass fractionators operation, pulp/paper mills operation, cellulosic ethanol refineries operation, and/or sugar cane mill operations.

33. A method for evaluation and processing of lignin, comprising the steps of:
providing lignin from at least two sources, wherein the composition of said lignin is unknown;
analyzing each of said lignin from at least two sources;
determining said composition of each of said lignin from said at least two sources;
using said determination of said composition of each of said lignin from said at least two sources to predict at least one biobased product;
determining said at least one of biobased product to produce; and
blending said lignin in defined proportions and amounts from said at least two sources to produce said at least one biobased product.

34. A method for evaluation of biomass feedstock, comprising the steps of:
providing lignin from a source comprising at least one biomass of woody plant biomass, agricultural plant biomass, cultivated plant biomass, kraft pulping biomass, sulfite pulping biomass, soda pulping biomass, cellulosic ethanol refinery biomass, sugarcane mill biomass, lignin residue biomass, and waste biomass, wherein the composition of said lignin is unknown;
analyzing said lignin by molecular spectroscopy;
analyzing said lignin biomass by physical-chemical analysis;
providing experimental data related to the molecular structure of said lignin;
performing regression analysis of said experimental data; and
determining said composition of said lignin;
wherein said step of determining said composition of said lignin provides a prediction of at least one biobased product produced from said lignin and said prediction has an accuracy of about 85% to about 100%.
